(19) United States
(12) Patent Application Publication
(10) Pub. No.: US 2015/0018667 A1
(43) Pub. Date: Jan. 15, 2015

(54) PROMOTING TRANSCRANIAL DIRECT CURRENT STIMULATION OR TRANSCRANIAL MAGNETIC STIMULATION USING TEMPERATURE-INDUCED SYNAPTIC MODULATION

(71) Applicant: THE UNITED STATES OF AMERICA, as represented by the Secretary, Dept. of Health and Human Services, Bethesda, MD (US)

(72) Inventors: Thomas C. Radman, Takoma Park, MD (US); Mark Hallett, Bethesda, MD (US)

(21) Appl. No.: 14/375,358

(22) PCT Filed: Jan. 29, 2013
(86) PCT No.: PCT/US2013/023672

§ 371 Date: Jul. 29, 2014

Related U.S. Application Data
(60) Provisional application No. 61/592,271, filed on Jan. 30, 2012.

(51) Int. Cl. A61N 2/00; A61B 5/0476; A61F 7/00; A61N 1/04; A61N 1/20; A61B 5/055; A61B 5/0488

(52) U.S. Cl. CPC A61N 2/002; A61N 2/006; A61B 5/055; A61B 5/0476; A61B 5/0488; A61N 1/0456; A61N 1/0492; A61N 1/20; A61F 7/0053; A61F 2007/0002
USPC 600/411; 600/9; 600/544; 600/546; 607/45; 607/3

(57) ABSTRACT

Disclosed herein are representative embodiments of methods, systems, and apparatus for enhancing or diminishing synaptic strength. Embodiments of the disclosed methods, systems, and apparatus can be used, for example, to complement the change in synaptic strength from transcranial direct current stimulation (“tDCS”) or transcranial magnetic stimulation (“TMS”) systems. One exemplary embodiment disclosed herein is a flexible housing having a top surface and a bottom surface. The flexible housing of this embodiment comprises a recessed cavity on the bottom surface that is configured to at least partially enclose an electrode of a transcranial direct current stimulator system. The flexible housing of this embodiment further comprises one or more apertures configured to provide access to the recessed cavity when the electrode is positioned within the recessed cavity. The flexible housing can further comprise one or more heating or cooling elements that can be selectively activated before, during, and/or after tDCS or TMS stimulation.

This application claims the benefit of U.S. Provisional Application No. 61/592,271, filed Jan. 30, 2012, which is incorporated herein by reference in its entirety.

### FIELD

This application relates generally to the field of modulating electrical and magnetic stimulation efficacy, or modulating synaptic strength, using temperature. Certain embodiments additionally involve transcranial direct current stimulation, transcranial magnetic stimulation, electro-convulsive therapy, or any other electrical or magnetic stimulation technique applied to the body. In some example embodiments, tissue that is not intended to be targeted by an electrical or magnetic treatment is cooled in order to decrease the cellular activity in the non-targeted tissue, thus increasing the focality of those treatments.

### SUMMARY

Disclosed below are representative embodiments of methods, systems, and apparatus used to stimulate synaptic transmission in subjects. Embodiments of the disclosed methods, systems, and apparatus can be used, for example, to promote, inhibit or further complement the neural stimulation or change in synaptic strength resulting from transcranial direct current stimulation (“tDCS”), transcranial magnetic stimulation (“TMS”), electro-convulsive therapy, (“ECT”) or variations of these methods.

Some of the embodiments disclosed herein comprise a device for use with a transcranial direct current stimulator system, the device comprising a flexible housing having a top surface and a bottom surface. The flexible housing can include a recessed cavity on the bottom surface, the recessed cavity being configured to at least partially enclose an electrode of the transcranial direct current stimulator system when the electrode is positioned within the recessed cavity. The flexible housing can further include one or more apertures configured to provide access to the recessed cavity when the electrode is positioned within the recessed cavity. In certain embodiments, the flexible housing further comprises one or more heating or cooling elements disposed in a body of the flexible housing. In some implementations, the one or more heating or cooling elements are disposed adjacent to the bottom surface of the flexible housing, adjacent to a bottom surface of the recessed cavity, or both adjacent to the bottom surface of the flexible housing and adjacent to the bottom surface of the recessed cavity. In certain implementations, the one or more heating or cooling elements comprise one or more electrically conductive elements that produce heat when an electrical current is applied to the electrically conductive elements. In some implementations, the one or more heating or cooling elements comprise one or more fluid conduits disposed in the body of the flexible housing. The fluid conduits can be fluidly connected to a system configured to heat or cool fluid and pump the heated or cooled fluid through the fluid conduits. In certain implementations, the one or more heating or cooling elements comprise one or more light emitting diode (“LED”) elements. In further implementations, the one or more heating or cooling elements comprise one or more ultrasound transducers. In certain implementations, the recessed cavity is configured so that a bottom surface of the electrode of the transcranial direct current stimulator system is coplanar or substantially coplanar with the bottom surface of the flexible housing when the electrode is positioned within the recessed cavity. In some implementations, the electrode of the transcranial direct current stimulator system is a sponge electrode. In certain implementations, the flexible housing is manufactured at least in part of latex, foam laminate, rubber, or silicone.

Certain embodiments disclosed herein comprise a method of treating a neurological condition or treating the neural effects of a condition (e.g., a cardiovascular condition, such as a stroke) wherein an electrode of a transcranial direct current stimulator system is positioned into a recessed cavity of a flexible housing; the flexible housing along with the electrode of the transcranial direct current stimulation system are placed against a region of the skull of a subject adjacent to a target region of the subject's cerebral cortex; one or more heating elements in the flexible housing are activated, thereby causing heat to be conducted from the flexible housing to the region of the skull of the subject adjacent to the target region; and the electrode of the transcranial direct current stimulation system is activated, thereby causing an electric current to be generated in the target region. In certain implementations, the one or more heating elements in the flexible housing are activated before the electrode of the transcranial direct current stimulation system is activated. Further, the one or more heating elements in the flexible housing can also be deactivated before the electrode of the transcranial direct current stimulation system is activated. In other implementations, the one or more heating elements in the flexible housing are deactivated at the same time or after the electrode of the transcranial direct current stimulation system is activated. In some implementations, the method is performed to treat a neurological condition (e.g., depression, Parkinson's disease, Alzheimer's disease, autism, post-traumatic stress disorder (“PTSD”), addictive behavior, anxiety, dysthymia, dystonia, epilepsy, pain, obsessive compulsive disorder, or schizophrenia). In further implementations, the method is performed to treat the neural effects of a condition (e.g., a cardiovascular condition, such as a stroke).

Other embodiments disclosed herein comprise a device for use with a transcranial magnetic stimulator coil, the device comprising a flexible housing having a top surface and a bottom surface. The flexible housing can define an interior cavity, the interior cavity being configured to at least partially enclose the transcranial magnetic stimulator coil. Further, the flexible housing can further include one or more apertures configured to provide access to the interior cavity when the transcranial magnetic stimulator coil is positioned within the interior cavity. In certain embodiments, the flexible housing further comprises one or more heating or cooling elements disposed in a body of the flexible housing. In some implementations, the one or more heating or cooling elements are disposed between the interior cavity and the bottom surface of the flexible housing. In further implementations, the one or more heating or cooling elements comprise one or more fluid conduits disposed in the body of the flexible housing. The fluid conduits can be fluidly connected to a system configured to heat or cool fluid and pump the heated or cooled fluid through the fluid conduits. In some implementations, the flexible housing has substantially the same shape as the transcranial magnetic stimulator coil. In certain implementations, the magnetic stimulator coil is one of a circular coil, skull-cap-shaped coil, double cone coil, slinky coil, H-coil, iron-core coil, circular crown coil, or figure-8 coil. In some implementations, the flexible housing is manufactured at least in part of latex, foam laminate, rubber, or silicone.

Further embodiments disclosed herein comprise another method of treating a neurological or cardiovascular condition wherein a transcranial stimulator coil is positioned into an interior cavity of a flexible housing, thereby at least partially enclosing the transcranial stimulator coil; the flexible housing is placed against a region of the skull of a subject adjacent to a target region of the subject's cerebral cortex; one or more heating elements in the flexible housing are activated, thereby causing heat to be conducted from the flexible housing to the region of the skull of the subject adjacent to the target region; and the transcranial magnetic stimulator coil is activated, thereby causing an electric current to be induced in the target region. In some implementations, the one or more heating elements in the flexible housing are activated before the transcranial magnetic stimulator coil is activated. In certain implementations, the one or more heating elements in the flexible housing are also deactivated before the transcranial magnetic stimulator coil is activated. In other implementations, the one or more heating elements in the flexible housing are deactivated at the same time or after the transcranial magnetic stimulator coil is activated. In further implementations, the method is performed to treat a neurological condition (e.g., depression, Parkinson's disease, Alzheimer's disease, autism, post-traumatic stress disorder (“PTSD”), addictive behavior, anxiety, dysthymia, dystonia, epilepsy, pain, obsessive compulsive disorder, or schizophrenia). In other embodiments, the method is performed to treat a cardiovascular condition (e.g., a stroke). In still other embodiments, the method is performed for research purposes (e.g., to determine the effect of potentiation on an area of the brain).

In further embodiments described herein, a method is disclosed that comprises, for a first period of time, increasing a temperature of a region of the cranium of a subject from a base line temperature to an elevated temperature, the region of the cranium of the subject being adjacent to a target region of the cerebral cortex of the subject, and sustaining the elevated temperature at the region on the cranium of the subject. The method further comprises, for a second period of time, activating a transcranial direct current stimulation electrode or a transcranial magnetic stimulator coil positioned at or adjacent to the region of the cranium of the subject, thereby electrically or electromagnetically stimulating the target region of the cerebral cortex of the subject, and sustaining the activation of the transcranial direct current stimulation electrode or the transcranial magnetic stimulator coil. In certain implementations, the first period of time ends before the second period of time begins. In further implementations, the first period of time and the second period of time at least partially overlap. In other implementations, the second period of time ends before the first period of time begins. In some implementations, the act of activating comprises activating the transcranial direct current stimulation electrode, and the act of increasing the temperature of the region of the cranium of the subject is performed using a flexible housing positioned on the region of the cranium of the subject, the flexible housing further including a recessed cavity in which the transcranial direct current stimulation electrode is positioned. In certain implementations, the method further comprises decreasing the temperature of the region of the cranium of the subject from the elevated temperature before the transcranial direct current stimulation electrode or the transcranial magnetic stimulator coil is activated. In some implementations, the method further comprises decreasing the temperature of the region of the cranium of the subject from the elevated temperature while the transcranial direct current stimulation electrode or the transcranial magnetic stimulator coil is activated. In still further implementations, the temperature of the region of the cranium of the subject is decreased from the base line temperature to a cooled temperature before the first period of time. In certain implementations, the act of increasing the temperature of the region of the cranium of the subject and the act of activating of the transcranial direct current stimulation electrode or the transcranial magnetic stimulator coil are performed concurrently. In some implementations, the act of increasing the temperature of the region of the cranium of the subject comprises activating one or more electrical heating elements in a flexible housing positioned on the region of the cranium of the subject. In certain implementations, the act of increasing the temperature of the region of the cranium of the subject comprises circulating a fluid at or above the elevated temperature through conduits in a flexible housing positioned on the region of the cranium of the subject. In some implementations, the act of increasing the temperature of the region of the cranium of the subject comprises activating one or more light-emitting diodes (“LEDs”) in a flexible housing positioned on the region of the cranium of the subject. In certain implementations, the act of increasing the temperature of the region of the cranium of the subject comprises activating one or more ultrasound transducers in a flexible housing positioned on the region of the cranium of the subject. In some implementations, the first period of time is 20 minutes or less. Further, the elevated temperature can be between 37.5° C. and 39° C. In certain implementations, the act of increasing the temperature of the region of the cranium of the subject comprises increasing the temperature at a rate between 2° C./minute and 5° C./minute. In some implementations, the method is performed as part of a treatment for depression, stroke recovery, Parkinson's disease, Alzheimer's disease, autism, post-traumatic stress disorder (“PTSD”), addictive behavior, anxiety, dysthymia, dystonia, epilepsy, pain, obsessive compulsive disorder, or schizophrenia.

In still other embodiments, a method is disclosed that comprises, for a first period of time, decreasing a temperature of a region of the cranium of a subject from a base line temperature to a cooled temperature, the region of the cranium of the subject being adjacent to a target region of the cerebral cortex of the subject, and sustaining the cooled temperature at the region on the cranium of the subject. The method further comprises, for a second period of time, activating a transcranial direct current stimulation electrode or a transcranial magnetic stimulator coil positioned at or adjacent to the region of the cranium of the subject, thereby electrically or electromagnetically stimulating the target region of the cerebral cortex of the subject, and sustaining the activation of the transcranial direct current stimulation electrode or the transcranial magnetic stimulator coil. In certain implementations, the first period of time ends before the second period of time begins. In some implementations, the first period of time and the second period of time at least partially overlap. In certain implementations, the second period of time ends before the first period of time begins. In some implementations, the method further comprises, increasing the temperature of the region of the cranium of the subject from the cooled temperature before the activating of the transcranial direct current stimulation electrode or the transcranial magnetic stimulator coil. In certain implementations, the method further comprises increasing the temperature of the region of the cranium of the subject from the cooled temperature while the transcranial direct current stimulation electrode or the transcranial magnetic stimulator coil are activated. In still further implementations, the temperature of the region of the cranium of the subject is increased from the base line temperature to an elevated temperature before the first period of time. In some implementations, the act of decreasing the temperature of the region of the cranium of the subject and the act of activating of the transcranial direct current stimulation electrode or the transcranial magnetic stimulator coil are performed concurrently. In further implementations, the act of decreasing the temperature of the region of the cranium of the subject comprises circulating a fluid at or below the cooled temperature through conduits in a flexible housing positioned on the region of the cranium of the subject. In some implementations, the method is performed as part of a treatment for depression, stroke recovery, Parkinson's disease, Alzheimer's disease, autism, post-traumatic stress disorder (“PTSD”), addictive behavior, anxiety, dysthymia, dystonia, epilepsy, pain, obsessive compulsive disorder, or schizophrenia.

In other embodiments described herein, a method is disclosed that comprises inducing release of intracellular calcium in cells in a target region of the cerebral cortex of a subject by increasing the temperature of the target region to an elevated temperature above a normothermic temperature for the target region; and inducing absorption of extracellular calcium in the cells in the target region by electrically stimulating the cells with an external stimulation device. In some implementations, the external stimulation device comprises a transcranial direct current stimulator device, and the act of electrically stimulating the cells comprises activating an electrode of the transcranial direct current stimulator system as the electrode is positioned on a surface of the head of the subject adjacent to the target region. In other implementations, the external stimulation device comprises a transcranial magnetic stimulator coil, and the act of electrically stimulating the cells comprises activating the transcranial magnetic stimulator coil as the transcranial magnetic stimulator coil is positioned on or next to a surface of the head of the subject adjacent to the target region. In some implementations, the act of inducing the release of the intracellular calcium further comprises maintaining the temperature of the target region at the elevated temperature for a period of time; and decreasing the temperature of the region to a temperature below the elevated temperature. In certain implementations, the elevated temperature is between 37.5° C. and 39°. In some implementations, the act of increasing the temperature of the target region, the act of maintaining the temperature of the target region, and the act of decreasing the temperature of the target region are performed before the cells are electrically stimulated. In further implementations, the act of increasing the temperature of the target region, the act of maintaining the temperature of the target region, and the act of decreasing the temperature of the target region are performed while the cells are electrically stimulated.

Other embodiments disclosed herein include a method comprising: inhibiting release of intracellular calcium in cells in a first region of the cerebral cortex of a subject by decreasing the temperature of the first region to a reduced temperature below a normothermic temperature for the first region; and inducing absorption of extracellular calcium in the cells in a second region of the cerebral cortex of the subject by electrically stimulating the cells with an external stimulation device. In these embodiments, the decreased temperature at the first region serves to focus the effect of the electrical stimulation to the second region of the cerebral cortex. In certain implementations, the external stimulation device comprises a transcranial direct current stimulator device, and wherein the electrically stimulating the cells comprises activating an electrode of the transcranial direct current stimulator system as the electrode is positioned on a surface of the head of the subject adjacent to the second region. In particular implementations, the external stimulation device comprises a transcranial magnetic stimulator coil, and wherein the electrically stimulating the cells comprises activating the transcranial magnetic stimulator coil as the transcranial magnetic stimulator coil is positioned on or next to a surface of the head of the subject adjacent to the second region. In some instances, the decreasing the temperature of the first region is performed before the cells in the second region are electrically stimulated and/or the decreasing the temperature of the first region is performed while the cells in the second region are electrically stimulated.

Further embodiments disclosed herein comprise a method in which the temperature of a region of the cranium of a subject is cooled from a base line temperature to a cooled temperature, the region of the cranium of the subject being adjacent to a device implanted in the subject (e.g., an implanted brain stimulator). In certain implementations, a magnetic resonance imaging scan of the cranium of the subject is also performed while the cranium is cooled. The cooled temperature of the cranium of the subject can be maintained during at least a portion of the magnetic resonance imaging scan. Further, the cooled temperature can be maintained for a period sufficient to cause cells in the cerebral cortex of the subject to have a reduced reaction to stimuli produced by the magnetic resonance imaging.

Further exemplary embodiments disclosed herein include a method comprising: causing the temperature of a subject's cranium to be changed from a normothermic temperature to a modulated temperature above or below the normothermic temperature, wherein the temperature change is induced by a heating or cooling device placed on the subject's cranium; detecting a set of one or more electrical potentials in the subject's brain or muscles while the subject's cranium is at the modulated temperature, the electric potentials being evoked by a set of one or more electrical or magnetic stimuli; and performing a diagnostic procedure using the detected set of one or more electrical potentials. In particular implementations, the set of one or more electrical potentials comprises a first set of one or more electrical potentials, wherein the modulated temperature is a first modulated temperature, wherein the set of one or more electrical or magnetic stimuli comprises a first set of one or more electrical or magnetic stimuli, and the method further comprises causing the temperature of a subject's cranium to be changed from the first modulated temperature to a second modulated temperature; detecting a second set of one or more electrical potentials in the subject's brain or muscles while the subject's cranium is at the second modulated temperature, the second set of electric potentials being evoked by a second set of one or more electrical or magnetic stimuli; and performing the diagnostic procedure using the detected first and second sets of electrical potentials. In some instances, the modulated temperature is above the normothermic temperature, while in other instances, the modulated temperature is below the normothermic temperature. The heating or cooling device placed on the subject's cranium can be a transcranial magnetic stimulator coil or transcranial direct current stimulator.

The foregoing and other objects, features, and advantages of the invention will become more apparent from the following detailed description, which proceeds with reference to the accompanying figures.

### BRIEF DESCRIPTION OF THE DRAWINGS

FIG. 1 is a perspective view of an exemplary system comprising a heating or cooling pad integrated with a tDCS electrode.

FIG. 2 is a perspective view of the embodiment of FIG. 1 being placed on the head of a subject.

FIG. 3 is a perspective view of another embodiment similar to that of the embodiment of FIG. 1 being placed on the head of a subject.

FIG. 4 is a top view of the exemplary embodiment shown in FIG. 1.

FIG. 5 is a cross-sectional side view of the exemplary embodiment shown in FIG. 1.

FIG. 6 is a bottom view of the exemplary embodiment shown in FIG. 1

FIG. 7 is a top view of an embodiment similar to the embodiment shown in FIG. 1 in which an electrical heating element is disposed in the body of the pad housing.

FIG. 8 is a cross-sectional side view of the exemplary embodiment shown in FIG. 7 along line 8-8 from FIG. 7.

FIG. 9 is a schematic diagram of an exemplary circuit that can be used to control and regulate an electrical heating element, such as the electrical heating element illustrated in FIGS. 7 and 8.

FIG. 10 is a top view of an embodiment similar to the embodiment shown in FIG. 1 in which a fluid conduit is disposed in the body of the heating or cooling pad housing.

FIG. 11 is a cross-sectional side view of the exemplary embodiment shown in FIG. 10 along line 10-10 from FIG. 10.

FIG. 12 is a cross-sectional side view of an embodiment similar to the embodiment shown in FIG. 1 in which one or more LED elements are disposed in the body of the heating or cooling pad housing.

FIG. 13 is a perspective view of an exemplary system comprising a heating or cooling pad integrated with a TMS coil.

FIG. 14 is a perspective view of the embodiment of FIG. 13 being placed on the head of a subject.

FIG. 15 is a top view of the exemplary embodiment shown in FIG. 13.

FIG. 16 is a cross-sectional side view of the exemplary embodiment shown in FIG. 13 along line 15-15 from FIG. 15.

FIG. 17 is a top view of an embodiment similar to the embodiment shown in FIG. 13 in which a fluid conduit is disposed in the body of the heating or cooling pad housing.

FIG. 18 is a cross-sectional side view of the exemplary embodiment shown in FIG. 17.

FIG. 19 is a perspective view of an exemplary system comprising a heating or cooling pad that is separate from a TMS coil.

FIG. 20 is a perspective view of the embodiment of FIG. 19 being placed on the head of a subject.

FIG. 21 is a top view of the exemplary embodiment shown in FIG. 19.

FIG. 22 is a cross-sectional side view of the exemplary embodiment shown in FIG. 21 along line 22-22 from FIG. 21.

FIG. 23 is a cross-sectional side view of another embodiment of a heating or cooling housing in which the material of the housing serves as the heating or cooling element and which includes an inner cavity in which a TMS coil can be located.

FIG. 24 is a cross-sectional side view of an embodiment similar to the embodiment shown in FIG. 1 in which one or more ultrasound elements are disposed in the body of the housing.

### DETAILED DESCRIPTION

### I. General Considerations

Disclosed herein are representative embodiments of methods, systems, and apparatus for stimulating synaptic activity in a subject's brain. The described methods, systems, and apparatus should not be construed as limiting in any way. Instead, the present disclosure is directed toward all novel and nonobvious features and aspects of the various disclosed embodiments, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present or problems be solved. Additionally, as used herein, the term “and/or” means any one item or combination of items in the phrase.

Furthermore, although the operations of some of the disclosed methods are described in a particular, sequential order for convenient presentation, it should be understood that this manner of description encompasses rearrangement, unless a particular ordering is required by specific language set forth below. For example, operations described sequentially may in some cases be rearranged or performed concurrently. Furthermore, while any of the disclosed methods can be performed using the tDCS or TMS devices described herein, they can also be performed using conventional tDCS, TMS, or other magnetic or electrical stimulation systems. Moreover, for the sake of simplicity, the attached figures may not show the various ways in which the disclosed methods, systems, and apparatus can be used in conjunction with other systems, methods, and apparatus.

### II. Introduction to Disclosed Technology

It has been observed that the application of heat to the mammalian brain can affect the synaptic activity in the brain. It has been observed, for example, that when the temperature of brain tissue from a test mammal is increased temporarily (e.g., up to 38.5° C. for about 15 minutes), synaptic strength initially decreases. This decrease in synaptic transmission is understood to be caused by activation of adenosine receptors. Upon the removal of the heat, however, synaptic strength is potentiated well beyond their base line levels. Additionally, it has been observed that the resulting increase in synaptic transmissions caused by the application and removal of heat is long term—lasting, for example, more than one hour. This synaptic potentiation is understood to be a result of increased cyclic adenosine monophosphate (“cAMP”) levels induced by the heat. The increased cAMP levels cause the release of intracellular calcium in the cells of the subject's brain, which in turn activates calcium-sensitive proteins that increase synaptic strength. Thus, it is currently understood that when sufficient heat is applied to a subject's cranium and subsequently removed, synaptic activity can be potentiated in the cerebral cortex of the subject. Furthermore, in some instances, this heat-induced potentiation can be long lasting (e.g., lasting more than one hour). Additionally, in some instances, synaptic activity can be potentiated during the application of heat to the subject's cranium, and not just after the heat removed.

The rate of temperature change can also affect the degree and duration of the heat-induced potentiation. For example, substantial and long-lasting potentiation has been observed in mammal brains when the rate of temperature change is substantially 3.5° C./min. It is to be understood, however, that other rates are possible, including faster rates of change or slower rates of change (both of which may result in improved potentiation, depending on the subject and application).

Potentiation caused by the heat is understood to use a different potentiation modality than potentiation caused by transcranial direct current stimulation (“tDCS”) or by transcranial magnetic stimulation (“TMS”). Specifically, in contrast to the intracellular calcium that is activated and released as a result of heat-induced potentiation, tDCS and TMS cause an increase in the cellular absorption of extracellular calcium. The increased influx of extracellular calcium is believed to be caused by N-methyl D-aspartate (“NMDA”) receptors that are activated by the electric current from the tDCS electrode or induced by the TMS coil. It has been observed, however, that NMDA receptors do not influence temperature-mediated potentiation, further suggesting that heat-induced potentiation operates with a unique potentiation modality.

The differing potentiation mechanisms between tDCS (or TMS) and heat allow the two modalities to complement each other. By using both heat-induced potentiation and potentiation from either tDCS or TMS (or some other electro or electro-magnetic stimulation), greater therapeutic effects can be achieved than from using either modality alone. Accordingly, embodiments of the disclosed technology comprise apparatus, systems, and methods for causing both heat-induced potentiation and electric-current-induced potentiation (resulting from tDCS, TMS, or some other electro- or electro-magnetic stimulation device).

Additionally, it has been observed that the electrical conductivity of tissue is proportional to temperature. Thus, the increased tissue temperature resulting from the use of embodiments of the disclosed technology can also increase the conductivity of the tissue being treated. As a result, the effectiveness of the tDCS or TMS treatment can be further improved. Conversely, a decrease in tissue temperature can decrease the cellular activity in areas not intended to be targeted by the electrical or magnetic treatment, thus increasing the focality of those treatments. A further possible advantage of applying heat before, during and/or after tDCS and TMS treatment is the activation of heat shock protein. Heat shock protein is a central nervous system protein that can be activated by high temperatures. Transient receptor potential vanilloid (“TRPV”) heat sensitive proteins form channels that may be as permeable to calcium influx as NMDA channels.

Embodiments of the disclosed technology can be used to therapeutically treat a wide variety of diseases or conditions. For example, any of the embodiments disclosed herein can be used to treat cardiovascular conditions (e.g., a stroke) or neurological conditions (e.g., depression, Parkinson's disease, or Alzheimer's disease). More generally, combined temperature-induced synaptic modulation and synaptic modulation from tDCS (or TMS) can be used to treat any brain disorder or condition in which synaptic modulation is desirable. For example, embodiments disclosed herein can be used to potentiate or reduce synaptic activity, depending on the desired application. Further, embodiments disclosed herein can be used to treat a variety of disorders, such as autism, post-traumatic stress disorder (“PTSD”), addictive behaviors (including smoking, overeating, and drug addiction), anxiety disorders, dysthymia, dystonia, epilepsy, pain, obsessive compulsive disorder, schizophrenia, or others. Still further, combined temperature-induced synaptic modulation and tDCS (or TMS) can be used as a therapeutic treatment for non-neurological disorders. For instance, any of the disclosed embodiments can be also be used to treat tissue trauma, bone damage, osteoporosis, and pain.

Embodiments of the disclosed technology can also be used to help reduce the risk of heating, aberrant current flow, and/or mechanical vibration and movement in patients with medical device implants who are to receive (or who are receiving) magnetic resonance imaging (“MRI”) scans. For example, a patient may have a deep brain stimulator or metal plate implanted in their cranium. The pulsed RF or other frequency magnetic fields that are used during an MRI scan can create undesirable currents and heat in the device or plate that can potentially burn or otherwise adversely affect the surrounding tissue. To help mitigate these effects, the temperature of the patient's cranium can be modulated (e.g., cooled) using any of the suitable embodiments disclosed herein.

Any of the disclosed embodiments can also be used for diagnostic purposes. In certain embodiments, for example, the TMS coil or stimulator is operated (e.g., using a single non-repeating pulse, paired-pulses, or repeating pulses) to evoke electrical potential changes in a patient's brain or muscles (motor-evoked potentials), or to evoke a sensory response reported by the subject (e.g., phosphenes when the visual cortex is stimulated). These evoked responses can then be used for diagnostic purposes. For instance, evoked potentiations generated from the brain can be used as an indicator of aberrant brain function (e.g., caused by a wide variety of potential diseases or conditions of the brain). Further, in certain implementations, the pulse can be applied and the response monitored while a patient's cranium and cerebral cortex are at a variety of different temperatures. The temperatures can be modulated using embodiments of the disclosed technology. Further, different cell types and neurotransmitters are understood to respond to temperatures at different rates, thus modulating the response to evoked potentials. The responses can then be compared to baseline or expected responses in order to detect aberrations.

Any of the disclosed embodiments can also be used for other purposes, such as academic or research purposes. For instance, any of the disclosed embodiments can be used to study brain activity and function. As one example, an area of the brain can be potentiated using any of the disclosed techniques to help determine what role the area has in the function of the brain (e.g., potentiation of the prefrontal cortex can be performed to determine the effect on decision making, or potentiation of the pre-motor cortex can be performed to determine the effect on reaction times).

In general, molecules involved in long-term potentiation (“LTP”) may be classified as either mediators or modulators. NMDA channels, whose activation results in an influx of extracellular calcium that helps to induce LTP, are thus termed mediators. Modulators are molecules who by themselves are not sufficient to induce LTP, but in combination with an LTP mediator, serve to enhance the potentiation caused by the mediator itself. Heat can be used to activate LTP modulators, such as cAMP. Dopamine, for example, has been demonstrated to enhance the potentiation observed through the cAMP/PKA pathway. In depressed patients, dopamine is typically downregulated. Thus, heat can be used effectively to circumvent the downregulated dopaminergic system in depressed patients by directly releasing cAMP and further promoting tDCS-induced potentiation.

### III. Exemplary Embodiments and Methods of Use

Some of the embodiments disclosed herein comprise heating devices that are configured to be used together with a transcranial direct current stimulation (“tDCS”) electrode or a transcranial magnetic stimulation (“TMS”) coil. In use, the heating devices are operable to heat the cranium and scalp of the subject before, during, and/or after activation of the tDCS electrode or TMS coil. The heating of the cranium and scalp using the heating device is understood to activate adenosine receptors in the cells of the subject cerebral cortex, and the removal of the heat (or continued application of the heat) is understood to increase cyclic adenosine monophosphate (“cAMP”) levels, thereby causing the release of intracellular calcium and triggering heat-related potentiation. At the same time the heat is applied or after the heat is applied and removed, the tDCS electrode can be operated to apply a current to the brain (e.g., a cathodal current), or the TMS coil can be operated to deliver non-repeating or repeating magnetic pulses (e.g., using single-pulse stimulation, paired pulse stimulation, paired-associative stimulation (such as rapid-rate paired associative stimulation (rPAS) in which rTMS stimulation is paired with a further electrical stimulus (e.g., to the median nerve)), quadrapulse stimulation, low frequency TMS, high frequency TMS, Theta bursts, or other such pulse trains or patterns). For example, the TMS pulses can be generated in succession (rTMS) at high rates (e.g., 5 Hz or faster), which can increase brain excitability. Because the heating device operates using a different and complementary potentiation modality than the tDCS electrode or TMS coil, the overall potentiation that can be achieved using embodiments of the disclosed technology is greater than when the tDCS electrode or TMS coil are used alone.

In some of the embodiments disclosed herein, the device used with the tDCS electrode or TMS coil is configured to not only provide heating, but can also provide cooling to the scalp, thereby resulting in another form of synaptic modulation. In still further embodiments, the device provides only cooling. In such embodiments, the heating/cooling or cooling device can be used to reduce the temperature of the subject's cranium and thereby reduce the efficacy of synaptic transmissions. In such embodiments, For example, TMS pulses can be generated in succession (rTMS) at lower rates (e.g., approximately between 0.2 and 1 HZ), which can decrease brain excitability. The cooling device can be operated to cool the scalp of the subject either before, during, and/or after activation of the tDCS electrode or TMS coil.

The sections below describe various embodiments of the cooling and/or heating device that are configured for use with a tDCS electrode or with a TMS coil. The disclosed embodiments are not to be construed as limiting, however, as they comprise representative examples of a wide variety of devices. For instance, it is to be understood that the disclosed cooling and/or heating devices are not limited to any particular shape, size, material of manufacture, configuration, or heating (or cooling) mechanism.

Furthermore, although the embodiments described below are described as being used with a tDCS electrode or a TMS coil, it is to be understood that any of the embodiments can be adapted for use with other electrical or magnetic stimulation devices. For example, any of the disclosed embodiments can be adapted for use with cranial electrical stimulation (“CES”) systems, transcranial electrical stimulation (“TES”) systems, transcranial alternating current stimulation (“tACS”) systems, electro-convulsive therapy systems, or any other such stimulation systems or devices (e.g., arrays of one or more electrodes used for stimulation).

A. Embodiments for Use with a Transcranial Direct Current Stimulation Electrode

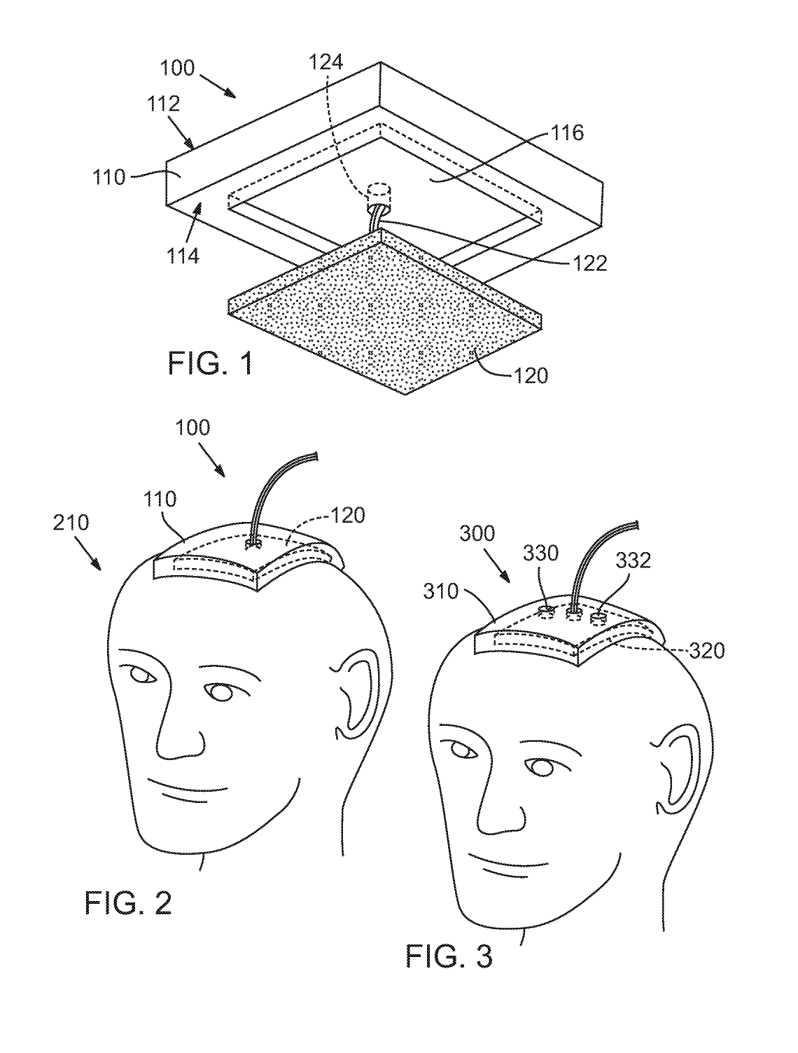

FIG. 1 is a perspective view showing an exemplary embodiment of a system 100 configured to apply heat to a subject's cranium and to apply transcranial direct current stimulation (“tDCS”). The exemplary system 100 comprises a housing 110 having a top surface 112 and a bottom surface 114. The bottom surface 114 of the housing 110 includes a recessed cavity 116 that is shaped and sized to receive a tDCS electrode 120. In the illustrated embodiment, the tDCS electrode 120 is a flexible sponge electrode and is coupled to a tDCS power generator (not shown) via one or more power wires 122. In the illustrated embodiment, the one or more power wires 122 extend through an aperture 124 formed in the recessed cavity 116 to the top side 112.

In particular embodiments, the housing 110 is configured to be flexible, thereby allowing it to be conformable to the cranium of a subject. For example, FIG. 2 shows the system 100 placed centrally on the head of a subject 210. As seen in FIG. 2, the housing 110 conforms to complement the curvature of the skull of the subject 210. As a result, the bottom surface 114 of the housing 110 is in direct contact with the surface of the scalp and effectively conducts heat to the skull. Additionally, as can also be seen in FIG. 2, the tDCS electrode 120 is also allowed to conform to the skull of the subject, thereby allowing the electrode 120 to more effectively stimulate the region of interest, typically a region of the cerebral cortex. Depending on the application, the tDCS electrode 120 can operate as an anode or cathode. For illustrative purposes, only a single electrode is shown in FIG. 2, though it is to be understood that the complementary electrode (also known as the reference electrode) would also be applied to the subject during tDCS. The main electrode can be placed in a variety of locations depending on the particular portion of the subject's brain to be stimulated (e.g., the primary motor cortex, the dorsolateral prefrontal cortex, or any portion or portions of the cerebral cortex). The reference electrode can also be placed in a variety of locations (e.g., elsewhere on the cranium or on the opposite shoulder or mastoid of the temporal bone from the region of interest).

In some embodiments, the tDCS electrode 120 is an electrode sponge and the effectiveness of the tDCS treatment is enhanced by moistening the tDCS electrode 120 with a liquid (e.g., saline or an electrolytic liquid). In such embodiments, one or more apertures can be formed in the housing through which liquid can be inserted or through which liquid can be removed. FIG. 3, for instance, is a perspective view of an embodiment of a system 300 in which housing 310 is similar to the housing 110 but includes a first electrode access aperture 330 and a second electrode access aperture 332. A suitable tube or liquid delivery mechanism (e.g., a pipette or syringe) can be inserted through the first electrode access aperture 330 and used to deliver liquid directly to sponge electrode 320. For example, the first electrode access aperture can be used to provide a saline drip to the sponge electrode, thereby preventing the drying out of the sponge electrode. Excess liquid can be removed via a suitable mechanism through the second electrode access 332.

In order to achieve the desired conformability, the housing 110 can be formed from a variety of moldable materials, such as latex, foam laminate, rubber, or silicone. The housing 110 can also be formed of a fabric or have a fabric covering. Furthermore, in certain embodiments, the material from which the housing is formed is selected to have a thermal conductivity that allows the housing 110 to be heated and cooled relatively quickly. Although the housing shown in FIG. 1 is shown as having a generally rectangular or square shape, the housing 110 can have a variety of shapes and thicknesses. For example, the housing 110 can be circular, skull-shaped, shaped to mimic the tDCS electrode or TMS coil, or have any other shape or configuration. As more fully illustrated below, the housing 110 can be formed to enclose one or more heating elements that can be activated to increase the temperature of the pad (e.g., the bottom surface 114 of the pad). In certain embodiments, the housing 110 can be formed to define one or more enclosures or cavities configured to securely house the one or more heating elements. The housing 110 can further include one or more additional apertures (not shown) through which a power cord or tube for supplying the one or more heating elements with power or circulating fluid extends. In other embodiments, the housing 110 is configured to enclose a battery source that is used to activate a circuit comprising one or more electrical heating elements.

In some embodiments, the housing 110 does not enclose any heating elements separate from the material forming the body of the housing. Instead, the body of the housing is formed of a material with a sufficiently low thermal conductivity such that that the housing can be preheated (e.g., using an external heat source, such as a microwave) and subsequently placed on the head of a subject, where it conducts heat to the subject's cranium and thereby increases the temperature of the adjacent region of the cerebral cortex.

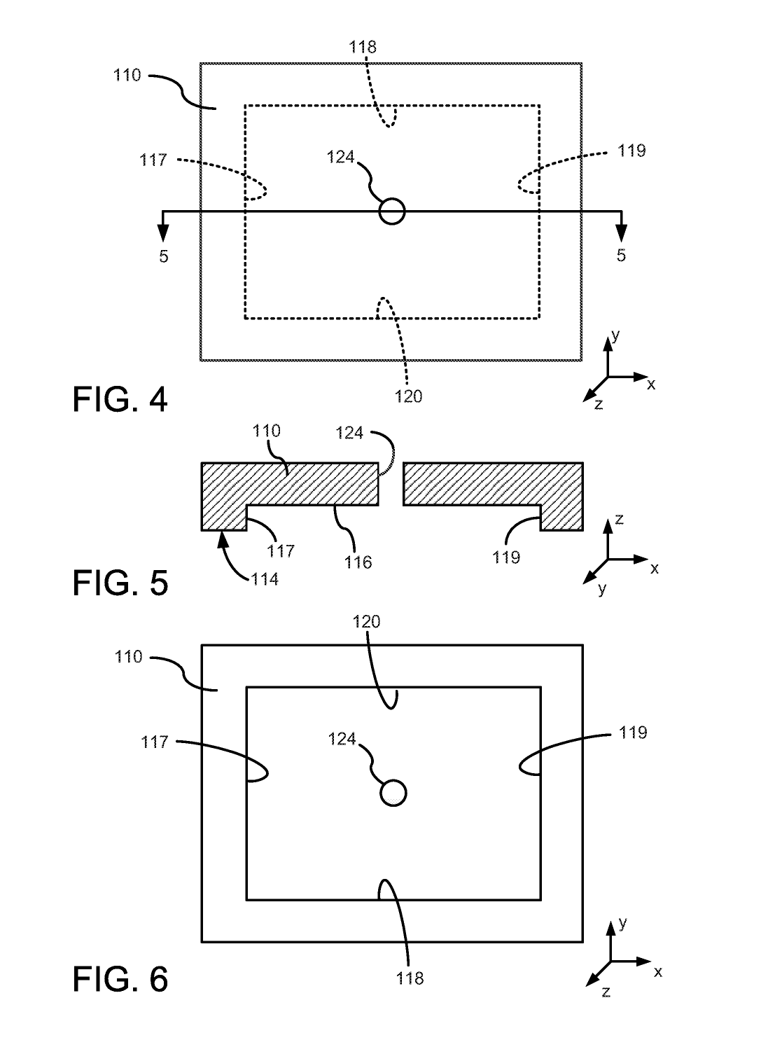

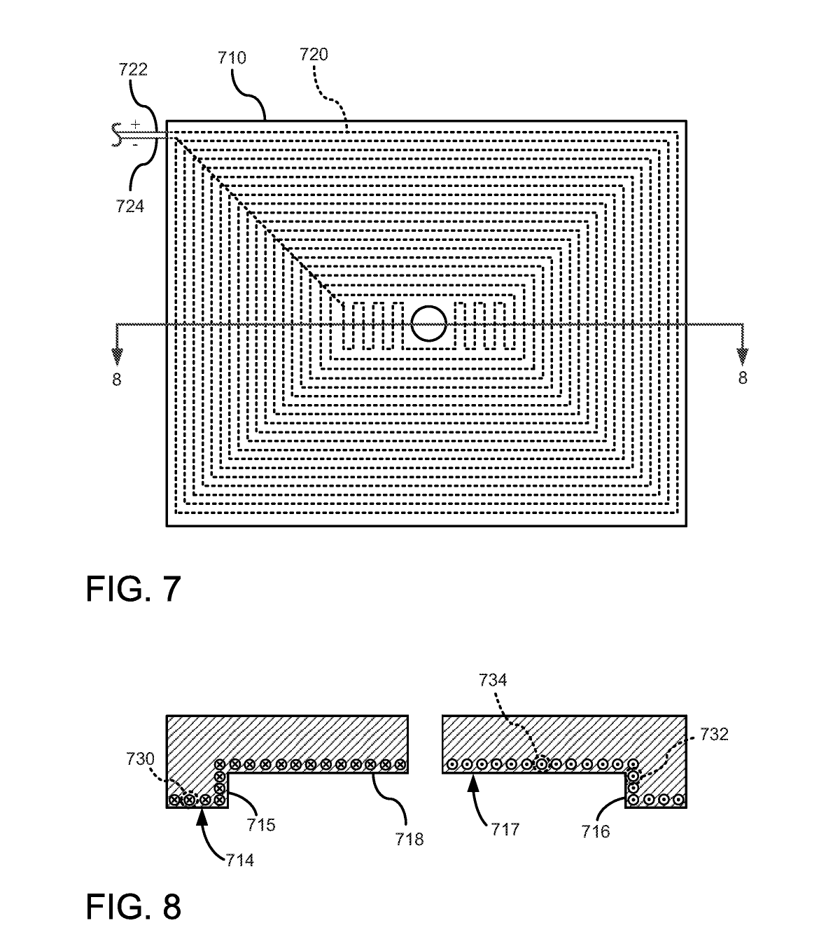

In the embodiment shown in FIG. 1, the housing 110 has a height dimension, a length dimension, and a width dimension. These dimensions are more fully illustrated in FIGS. 4-6. In particular, FIG. 4 is a top view of the housing 110, FIG. 5 is a cross-sectional side view of the housing 110, and FIG. 6 is a bottom view of the housing 110 (showing the housing 110 flipped from its position in FIG. 4). FIGS. 4-6 also illustrate the location of the recessed cavity 116, which includes cavity walls 117, 118, 119, 120. FIGS. 4-6 also illustrate aperture 124, which is shown as being centrally located, but can be located in other positions in the housing. In one exemplary embodiment, the housing 110 has a length dimension (shown as extending along the x-axis) of 9 cm, a width dimension (shown as extending along the y-axis) of 7 cm, and a height dimension (shown as extending along the z-axis) of 1.6 cm. Thus, in this embodiment, the height dimension is less than that of the length or width dimensions. Furthermore, in the illustrated embodiment, the recessed cavity 116 has a height that is selected to allow the bottom surface of the electrode 120 to be on or substantially on the same plane as the bottom surface 114 of the housing 110. The cross-sectional view shown in FIG. 5 shows the housing as being a solid material. As shown below in FIGS. 7-8, 10, 11, 12, and 23, the housing can include one or more heating elements that serve to create heat within the housing, thereby heating the bottom surface of the housing such that the housing conducts heat to the surface of the cranium of a subject, and thereby increases the temperature of the cells in the subject's brain (e.g., in the cerebral cortex).

FIGS. 7 and 8 illustrate one heating mechanism that can be used in embodiments of the disclosed technology. In particular, FIG. 7 is a top view of a housing 710, and FIG. 8 is a cross-sectional side view of the housing 710. The housing 710 of FIGS. 7 and 8 is similar to that of housing 110 but includes a heating coil 720. In the illustrated embodiment, the heating coil 720 is a single heating coil that winds from a first point 722 through a rectangular spiral of decreasing diameter, through a center portion, and to a second point 724 adjacent to the first point 722. Although only a single heating coil is shown in FIG. 7, one or more additional heating coils can be used. In the illustrated embodiment, the first point 722 and second point 724 are located next to each other so that they can extend through a single or adjacent apertures in the housing and so that the external wiring to the housing 710 is simplified. The heating coil can be formed of a variety of metals or alloys (e.g., Nichrome, iron-chrome, nickel-chrome, nickel-iron, nickel, stainless steel, molybdenum, tungsten, molybdenum silicide, and the like) and have a variety of dimensions and configurations (e.g., a wire shape, ribbon shape, braided wire shape, and the like). In general, the heating coil should exhibit a resistance that causes the heating coil to heat up when an electric current is applied to the coil.

As best shown in the cross-sectional view of FIG. 8, one or more windings of the heating coil 720 can be positioned within the housing so that they are near or adjacent to a bottom surface 714 of the housing 710. For example, representative winding 730 is located at a position just above the bottom surface 714. As also shown in the cross-sectional view of FIG. 8, one or more windings of the heating coil 720 can also be positioned so that they are near or adjacent to the walls 715, 716 and bottom surface 717 of recessed cavity 718. For example, representative windings 732, 734 are positioned just inside of the recessed cavity wall 716 and just above the bottom surface 717, respectively.

In other embodiments, at least a portion of the housing is made of a material that heats up in response to the application of an electrical current to the material itself, such as electrically conductive silicone rubber. For example, the entire housing can be formed of electrically conductive silicone rubber or only a portion adjacent to the bottom surface of the housing. The portion of the housing made of electrically conductive silicone rubber can then be coupled to a suitable circuit.

The heating coil can be powered by any suitable voltage source. For example, in some embodiments, an external AC-powered voltage source that is electrically isolated is used to protect from electrical shock hazards that may result from a single fault conditions. Or, in some embodiments, an internal DC-powered voltage source is used.

The operation of the heating coil (e.g., the temperature, operational state, and operational time) can be controlled by control circuitry that is located either external of the housing 710 (as in the illustrated embodiment) or internal to the housing. A wide variety of control circuitry can be used to control the heating coil. In general, however, the control circuitry is configured to selectively apply current to the heating coil to reach and maintain a desired operational temperature. Thus, the control circuitry is typically regulated by a thermistor and, in some implementations, is controlled by a microprocessor or microcontroller. The control circuitry can further include a timer and be configured to control both the one or more heating elements and the tDCS electrode or the TMS coil so that they operate according to any of the methods disclosed below (e.g., according to any of the methods described in Section III.C below).

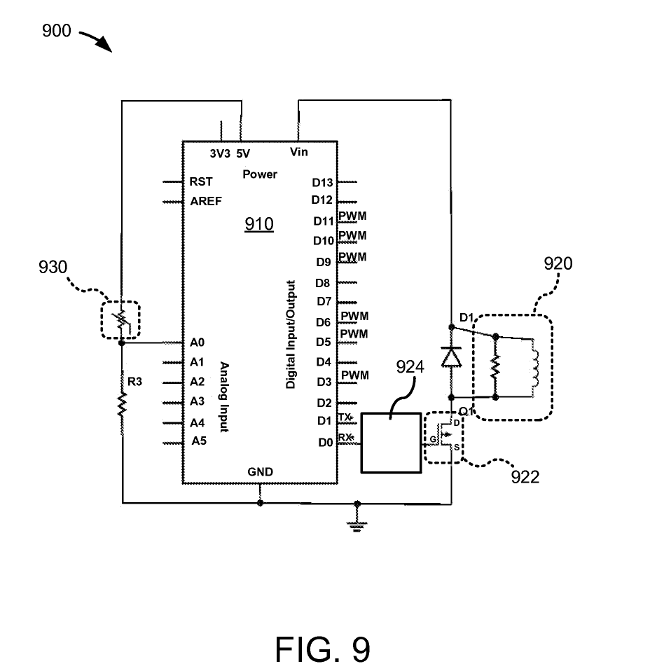

One exemplary circuit configuration for controlling the heating coil (or for selectively activating any of the disclosed heating elements in Sections III.A and III.B) is illustrated in FIG. 9. In particular, FIG. 9 is a circuit diagram 900 of an example circuit suitable for controlling the heating coil. In the example circuit, a microcontroller 910 (e.g., an Atmel ATmega328 microcontroller) selectively activates the heating element 920 in the housing 710. In FIG. 9, the heating element 920 is a heating coil shown as including a resistive component as well as an inductive component. The heating element 920 is selectively activated by controlling the gate at transistor 922, which is coupled to the microcontroller 910 through protective circuitry 924. The circuit diagram 900 also includes a thermistor 930 that can be used to monitor the temperature of either the heating element 920, some other portion of the housing 710, or the surface of the subject's cranium adjacent to the region of interest. Based on the resistance of the thermistor 930 (which indicates the temperature at the thermistor 930), the microcontroller 910 can be selectively shut off or activate the heating element 920. In this way, the thermistor 930 can be used to regulate the activation of the heating element 920 so that a desired temperature is maintained by the heating element when it is activated. The microcontroller 910 can further be programmed to selectively activate the heating element 920 for a period of time, and then selectively activate the electrodes of the tDCS system (or the coil of the TMS system) for another period (e.g., according to any of the methods described in Section III.C below). For instance, the electrodes of the tDCS system can be controlled through a signal from one of the digital input/outputs of the microcontroller 910. In other embodiments, the electrodes of the tDCS system are separately controlled. In still other embodiments, a microcontroller 910 can be configured to detect activation of the electrodes of the tDCS system (e.g., via a signal input into one of the digital input/outputs of the microcontroller) and to deactivate the heating element 920 upon activation of the electrodes. The example circuit shown in FIG. 9 should not be construed as limiting, as a wide variety of circuit configurations can be used to control the one or more heating elements.

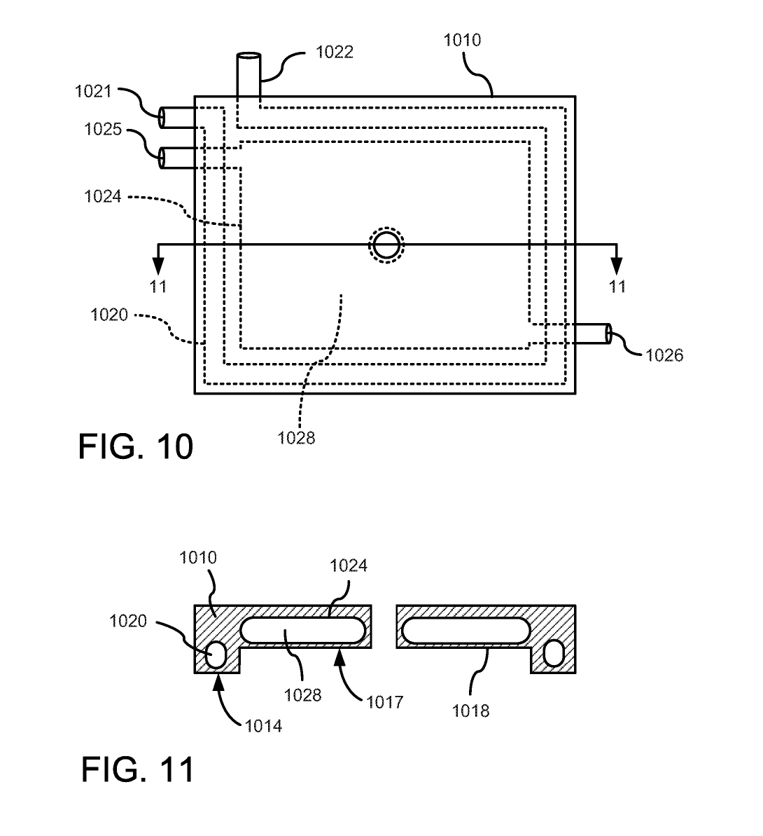

FIGS. 10 and 11 illustrate another heating mechanism that can be used in embodiments of the disclosed technology. In particular, FIG. 10 is a top view of a housing 1010, and FIG. 11 is a cross-sectional side view of the housing 1010. The housing 1010 of FIGS. 10 and 11 is similar to that of housing 110 but includes one or more conduits for circulating a fluid through one or more interior spaces of the housing 1010. The fluid can be a liquid (e.g., water or saline) or a gas (e.g., air). In the illustrated embodiment, for example, a first conduit 1020 extends along the perimeter of the interior of the housing 1010. The first conduit 1020 additionally includes an inflow side 1021 and an outflow side 1022. A second conduit 1024 extends into the central interior of the housing 1010 and includes a central chamber 1028. The second conduit 1024 additionally includes an inflow side 1025 and an outflow side 1026. Although two conduits are shown in the illustrated embodiments, more or fewer conduits can be implemented in the housing. The housing 1010 can be formed to include suitable spaces or channels for the conduits. The conduits themselves can be formed from a variety of materials. For example, the conduits can be formed from a synthetic material (e.g., rubber, silicone, or the like) or a suitable metal or alloy. Alternatively, in certain embodiments, the conduits do not have a separate conduit wall but are instead formed directly from the material from which the housing 1010 is manufactured. In other words, the channels and interior space of the housing 1010 itself serves as the conduit walls.

As best shown in the cross-sectional view of FIG. 11, the body of the first conduit 1020 is located near or adjacent to a bottom surface 1014 of the housing 1010. As also shown in FIG. 11, the central chamber 1028 of the second conduit 1024 is located near or adjacent to a bottom surface 1017 of recessed cavity 1018. The first and second conduits 1020, 1024 can be used to circulate a heated or cooled fluid (or heated or cooled air or other gas) through the housing, thereby changing the temperature of the housing to a desired temperature. To supply the heated or cooled fluid, the first and second conduits 1020, 1024 can be fluidly coupled to a variety of pumping or forced air systems with heating and/or cooling capabilities. In use, the first conduit 1020 can be used to alter the temperature of a subject's skull directly while second conduit 1024 and the central chamber 1028 can be used to alter the temperature of the electrode of the tDCS system. In turn, the heated or cooled electrode (which is typically moistened with liquid) can be used to alter the temperature of the subject's skull. Because some heat loss might occur in the electrode, the temperature of the fluid circulated through the second conduit 1024 may be different than the temperature of the fluid circulated through the first conduit 1020 (e.g., the temperature of the liquid or gas in the second conduit 1024 can be greater than the temperature of the liquid or gas in the first conduit 1020, or vice versa). One or more thermal sensors (e.g., a thermistor or other temperature sensing device) can be affixed to the bottom surface of the housing 1010 or placed on the scalp of the subject at or near the region being treated in order to monitor the temperature. The circulation and temperature and of the fluid being circulated through the first and second conduits 1020, 1024 can then be adjusted as appropriate in order to achieve the desired temperature changes.

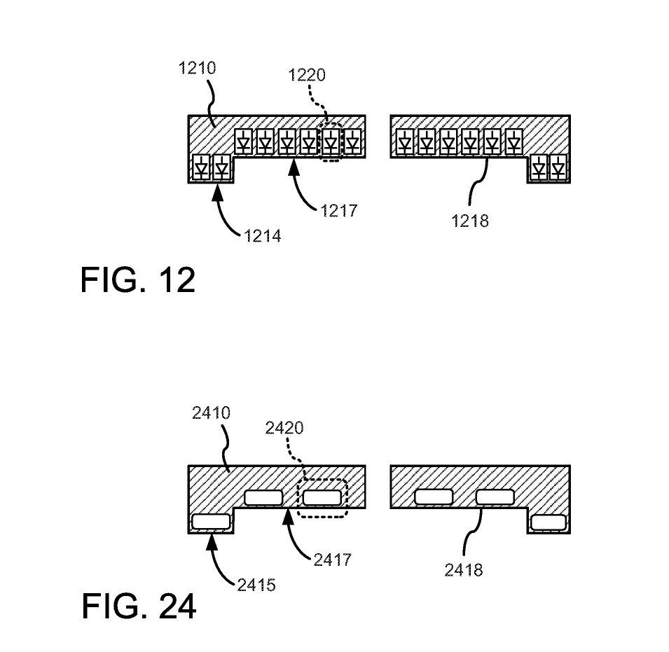

FIG. 12 illustrates another heating mechanism that can be used in embodiments of the disclosed technology. In particular, FIG. 12 is a cross sectional view of a housing 1210. The housing 1210 of FIG. 12 is similar to that of housing 110 but includes one or more LED elements, a representative one of which is shown as LED element 1220. The LED elements can be high-energy LED elements that emit heat as well as light when activated. Furthermore, the LED elements can be arranged in the housing 1210 so that the elements are located at or near a bottom surface 1214 of the housing 1210 and at or near a bottom surface 1217 of a recessed cavity 1218 of the housing that is shaped to receive the electrode of a tDCS system. The LED elements can be powered and controlled by a power system that selectively activates and deactivates the LED elements in order to reach and maintain a desired temperature. One or more thermal sensors (e.g., a thermistor or other temperature sensing device) can be affixed to the bottom surface of the housing 1210 or placed on the scalp of the subject at or near the region being treated in order to monitor the temperature. The LEDs can then be activated or deactivated as appropriate in order to achieve the desired temperature changes. A further possible advantage of this embodiment is that the cellular absorption of photons (such as photons from the LED elements) is understood to promote an increase in cellular respiration and intracellular calcium. Photon absorption can also decrease inflammation by a reduction of NF-kB and up-regulation of cytoprotective gene products such as superoxide dismutase, glutathione peroxidase, and heat shock protein 70. Other therapeutic effects might be realized from the penetration of photons into the brain. In other embodiments, other lighting elements are used in place of or in combination with the one or more LEDs (e.g., filament bulbs, halogen bulbs, and the like).

FIG. 24 illustrates another heating mechanism that can be used in embodiments of the disclosed technology. In particular, FIG. 24 is a cross sectional view of a housing 2410. The housing 2410 of FIG. 24 is similar to that of housing 110 but includes one or more ultrasound transducers, a representative one of which is shown as ultrasound transducer 2420. The ultrasound transducers can be configured to emit heat as well as ultrasonic waves when activated. Furthermore, the ultrasound transducers can be arranged in the housing 2410 so that the elements are located at or near a bottom surface 2414 of the housing 2410 and at or near a bottom surface 2417 of a recessed cavity 2418 of the housing that is shaped to receive the electrode of a tDCS system. The ultrasound transducers can be powered and controlled by a power system that selectively activates or deactivates the ultrasound transducers (either individually, in groups of two or more, or as an entire group) in order to reach and maintain a desired temperature. For example, by independently actuating groups of one or more ultrasound transducers surrounding the targeted stimulation area, the summation of ultrasound waves at the desired depth and location of heating can be controlled. Furthermore, in certain embodiments, the ultrasound transducers can be operated to mechanically move neural tissue without producing heat (e.g., by reducing the output of the transducers). One or more thermal sensors (e.g., a thermistor or other temperature sensing device) can be affixed to the bottom surface of the housing 2410 or placed on the scalp of the subject at or near the region being treatment in order to monitor the temperature. The ultrasound transducers can then be activated or deactivated as appropriate in order to achieve the desired temperature changes. In certain embodiments, the ultrasound transducers are operated to provide superficial heating or depth targeted heating. Whether superficial or depth targeted heating is performed can depend, for example, on what type of tDCS or TMS system is used and the depth of electrical/magnetic stimulation possible with such a system.

In other embodiments, and more generally, any form of diathermy can be used as the heating mechanism for embodiments of the disclosed technology. For example, the housing can be configured to comprise one or more transducers or other electrical components that generate radio-frequency electromagnetic waves and that can increase the temperature of an adjacent surface or of a targeted region beneath the surface (e.g., a targeted region of the cerebral cortex).

In another embodiment of the disclosed technology, the housing encloses one or more heating elements that produce heat from a chemical reaction in the heating elements. For example, the heating elements can be configured to produce an exothermic reaction when chemicals in the heating elements are mixed or react upon activation (e.g., crystallization of sodium acetate).

B. Embodiments for Use with a Transcranial Magnetic Stimulator Coil

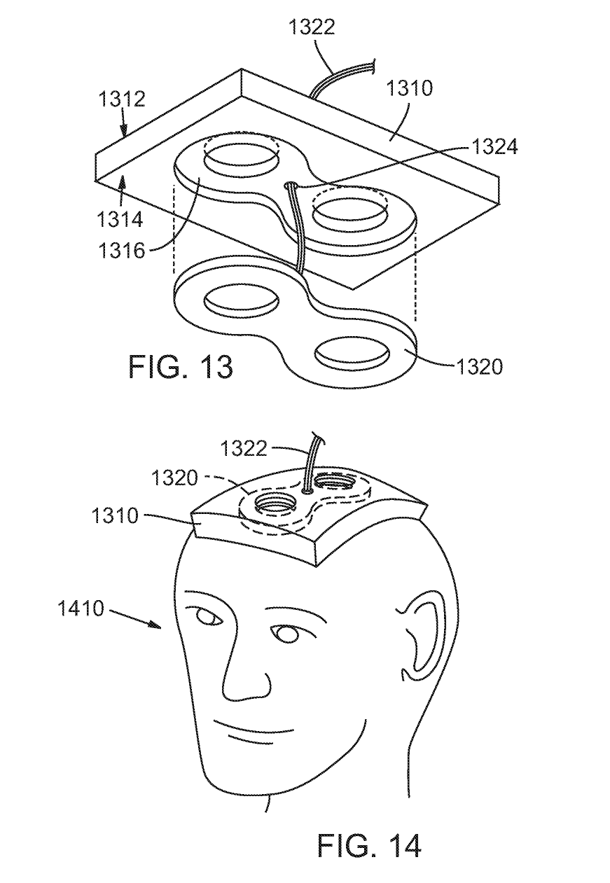

FIG. 13 is a perspective view showing an exemplary embodiment of a system 1300 configured to apply heat to a subject's cranium and to apply transcranial magnetic stimulation (“TMS”) using a transcranial magnetic stimulation coil. The exemplary system 1300 comprises a housing 1310 having a top surface 1312 and a bottom surface 1314. The bottom surface 1314 of the housing 1310 includes a recessed cavity 1316 that is shaped and sized to receive a TMS coil 1320. In the illustrated embodiment, the TMS coil 1320 is a figure-8 coil that is enclosed in a separate housing. Although a figure-8 coil is shown in FIG. 13, the housing 1310 can be configured to receive a wide variety of TMS coils (e.g., circular coils, skull-cap-shaped coils, double cone coils, slinky coils, H-coils, C-core coils, circular crown coils, or any other suitable TMS single coil or multiple coil configuration). In the illustrated embodiment, the TMS coil 1320 is coupled to a power generator (not shown) via one or more power wires 1322. In the illustrated embodiment, the one or more power wires 1322 extend through an aperture 1324 formed in the recessed cavity 1316 and extending through the top side 1312.

In particular embodiments, the housing 1310 is configured to be flexible, thereby allowing it to be conformable to the cranium of a subject. For example, FIG. 14 shows the housing 1310 placed centrally on the head of a subject 1410. As seen in FIG. 14, the housing 1310 conforms to complement the curvature of the skull of the subject 1410. As a result, the bottom surface 1314 of the housing 1310 is in direct contact with the surface of the skull and conducts heat to the skull in an effective manner. The housing 1310 can be constructed using any of the materials discussed above with respect to housing 110. The TMS coil 1320 can include one or more hinges or be formed of a flexible material that allows the TMS coil to similarly conform to the skull of the subject, thereby allowing the TMS coil to more effectively stimulate the region of interest. The TMS coil can be placed in a variety of locations depending on the particular portion of the subject's brain to be stimulated (e.g., the primary motor cortex, the dorsolateral prefrontal cortex, or any other portion or portions of the cerebral cortex). In use, the TMS coil 1320 can be used to apply a variety of different TMS pulse patterns with a wide variety of frequencies (e.g., single-pulse TMS, paired pulse TMS, paired-associative stimulation (such as rapid-rate paired associative stimulation (rPAS) in which TMS stimulation is paired with a further electrical stimulus (e.g., to the median nerve)), quadrapulse stimulation, low frequency TMS, high frequency TMS, Theta bursts, or other such pulse trains or patterns).

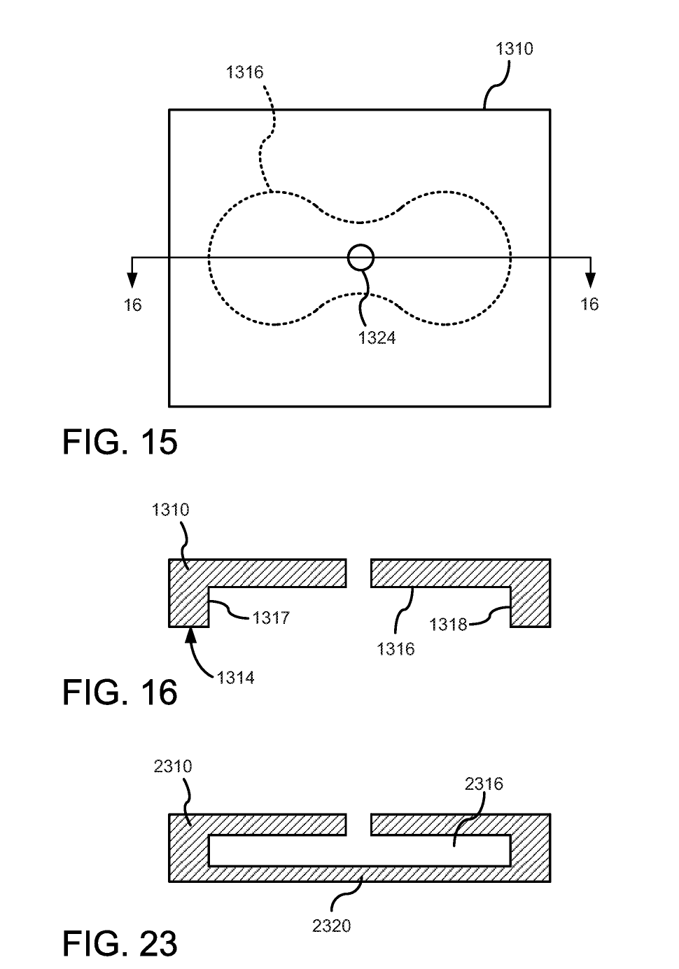

FIG. 15 is a top view of the housing 1310, and FIG. 16 is a cross-sectional side view of the housing 1310. FIGS. 15 and 16 illustrate the location of the recessed cavity 1316, which includes cavity walls 1317, 1318. FIGS. 15 and 16 also illustrate aperture 1324, which is shown as being centrally located, but can be located in other positions in the housing 1310. Although the housing shown in FIG. 15 is shown as having a generally rectangular or square shape, the housing 1310 can have a variety of shapes and thicknesses. For example, the housing 1310 can be circular, skull-shaped, shaped to mimic or substantially mimic the shape of the TMS coil, or have any other shape or configuration. Furthermore, although the recessed cavity 1316 is shown as having a shape that mimics the TMS coil, it can have a wide variety of shapes (e.g., a sleeve that allows the TMS coil to slide into place).

In the illustrated embodiment, the housing 1310 has dimensions that are larger than those of the TMS coil so that a portion of the bottom surface 1314 of the housing 1310 surrounds the periphery of the TMS coil. In general, the dimensions of the housing 1310 will vary from embodiment to embodiments depending on the shape and size of the TMS coil as well as the desired treatment region. In the illustrated embodiment, the recessed cavity 1316 has a height that is selected to allow the bottom surface of the TMS coil 1320 to be on or substantially on the same plane as the bottom surface 1314 of the housing 1310.

In other embodiments, the housing includes a cavity for the TMS coil that is entirely within the interior of the housing such that the bottom surface of the TMS coil is not exposed. FIG. 23, for example, is a cross-sectional side view of an exemplary housing 2310 similar to that of housing 110 but that has an interior cavity 2316 configured to receive the TMS coil. As illustrated in FIG. 23, a bottom portion 2320 of the housing 2310 separates the TMS coil from the subject's cranium. Because TMS operates by inducing currents in the subject's brain as a result of a changing electromagnetic field generated by the TMS coil, direct contact between the TMS coil and the skin of the subject cranium is typically not required (though it can be beneficial). The shape of the interior cavity 2316 can vary from embodiment to embodiment. For example, the interior cavity 2316 can have a shape that mimics the TMS coil, or can be shaped as a sleeve that allows the TMS coil to slide into place within the interior of the housing 2310.

The cross-sectional views of both FIGS. 16 and 23 show the respective housings 1610, 2310 as being a solid material. In such embodiments, the body of the housing can be formed of a material with a sufficiently low thermal conductivity such that that the housing can be preheated (e.g., using an external heat source, such as a microwave) and subsequently placed on the head of a subject, where it conducts heat to the subject's cranium and thereby increases the temperature of the adjacent region of the cerebral cortex. Or, in other embodiments, at least a portion of the housing is made of a material that heats up in response to the application of an electrical current to the material itself, such as electrically conductive silicone rubber. For example, the entire housing can be formed of electrically conductive silicone rubber or only a portion adjacent to the bottom surface of the housing. The portion of the housing made of electrically conductive silicone rubber can then be activated by the TMS coil or by a separate, external power source located away from the TMS coil.

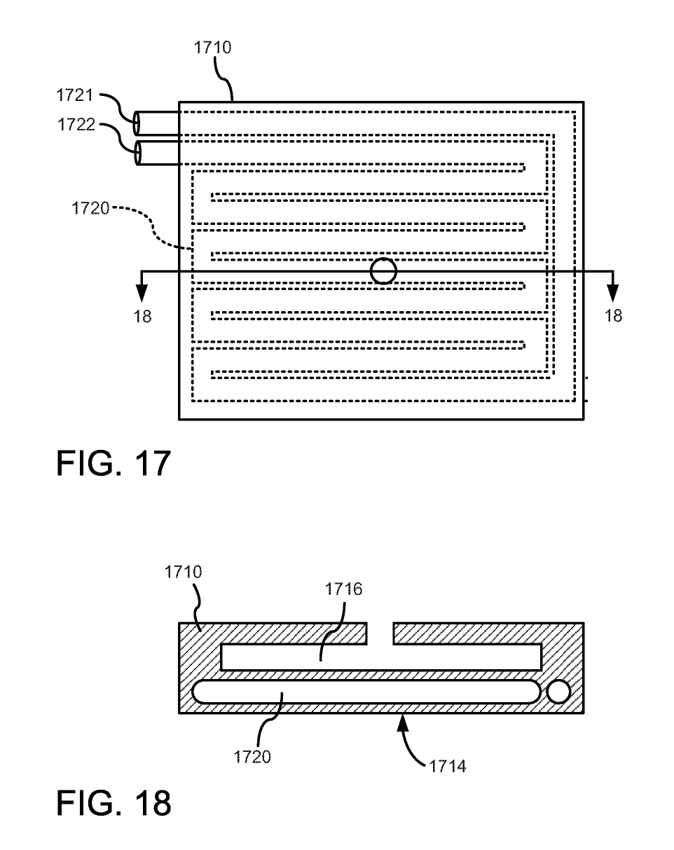

As shown below in FIGS. 17-18, some embodiments of the housing include one or more heating elements that serve to create heat within the housing, thereby heating the bottom surface of the housing such that the housing conducts heat to the surface of the cranium of a subject when placed on the subject's head. Because the TMS coil creates high-intensity time-varying magnetic fields, the heating elements of the pad desirably do not comprise any electrical components or circuits whose performance could be impacted by the changing fields.

FIGS. 17 and 18 illustrate one heating mechanism that can be used in embodiments of the disclosed technology. In particular, FIG. 17 is a top view of a housing 1710, and FIG. 18 is a cross-sectional side view of the housing 1710. The housing 1710 of FIGS. 17 and 18 is similar to that of housing 1510 but includes one or more conduits for circulating a fluid through one or more interior spaces of the housing 1710. In the illustrated embodiment, for example, a first conduit 1720 extends into and winds through the interior of the housing 1710. The first conduit 1720 additionally includes an inflow side 1721 and an outflow side 1722, which in the illustrated embodiment are located next to one another. Although only a single conduit is shown in FIGS. 17 and 18, one or more additional conduits can be manufactured into the housing 1710. The housing 1710 can be formed to include suitable spaces or channels for the one or more conduits. The conduits themselves can be formed from a variety of materials, as described above with respect to FIG. 10-11. Alternatively, in certain embodiments, the one or more conduits do not have a separate conduit wall but are instead formed directly from the material from which the housing 1710 is manufactured.

As best shown in the cross-sectional view of FIG. 18, the body of the first conduit 1720 is located between a bottom surface 1714 of the housing 1710 and an interior cavity 1716 in which the TMS coil is located. The first conduit 1720 can be used to circulate a heated or cooled fluid (e.g., a liquid or gas, such as air) through the housing 1710, thereby changing the temperature of the bottom surface 1714 of the housing to a desired temperature. To supply the heated or cooled fluid, the first conduit 1720 can be fluidly coupled to a variety of pumping or forced-air systems with heating and/or cooling capabilities. In use, the first conduit 1720 can be used to alter the temperature of a subject's skull prior to or during TMS treatment. One or more thermal sensors (e.g., a thermistor or other temperature sensing device) can be affixed to the bottom surface of the housing 1710 or placed on the scalp of the subject at or near the region being treated in order to monitor the temperature. The circulation and temperature and of the fluid being circulated through the first conduit 1720 can then be adjusted as appropriate in order to achieve the desired temperature changes.

In another embodiment of the disclosed technology, the housing encloses one or more heating elements that produce heat from a chemical reaction in the heating elements. For example, the heating elements can be configured to produce an exothermic reaction when chemicals in the heating elements are mixed or react upon activation (e.g., crystallization of sodium acetate). Furthermore, in some embodiments, the TMS coil itself generates heat during operation. Thus, the TMS coil can serve as a heating source for the pad 2110.

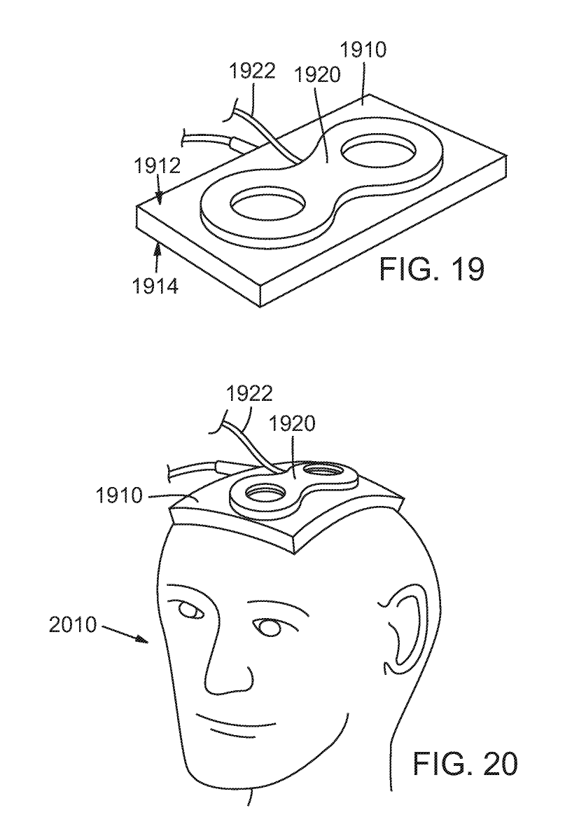

In other embodiments, the TMS coil is separate from the housing that provides heat or cooling to the subject's cranium. FIG. 19 is a perspective view showing an exemplary embodiment of a system 1900 in which a pad 1910 is separate from the TMS coil 1920. The pad 1910 comprises a top surface 1912 and a bottom surface 1914 and can be constructed and configured in any of the manners described above. In the illustrated embodiment, the TMS coil 1920 is a figure-8 coil. Although a figure-8 coil is shown in FIG. 19, any of the coils outlined above with respect to FIG. 13-14 can be used. In the illustrated embodiment, the TMS coil 1920 is coupled to a power generator (not shown) via one or more power wires 1922.

In particular embodiments, the pad 1910 is configured to be flexible, thereby allowing it to be conformable to the cranium of a subject. For example, FIG. 20 shows the pad 1910 placed centrally on the head of a subject 2010. As seen in FIG. 20, the pad 1910 conforms to complement the curvature of the skull of the subject 2010. As a result, the bottom surface 1914 of the pad 1910 is in direct contact with the surface of the scalp and effectively conducts heat to the skull. The TMS coil 1920 can then be placed on the top surface 1912 of the pad 1910 and used to apply any of a variety of different TMS pulse patterns.

The interior of the heating pad 1910 can be solid or can include one or more heating elements that serve to create heat within the body of the pad (e.g., one or more fluid conduits or chemical heating elements), thereby heating the bottom surface of the pad. Because the TMS coil creates high-intensity time-varying magnetic fields, the heating elements of the pad desirably do not comprise any electrical components or circuits whose performance could be impacted by the changing fields.

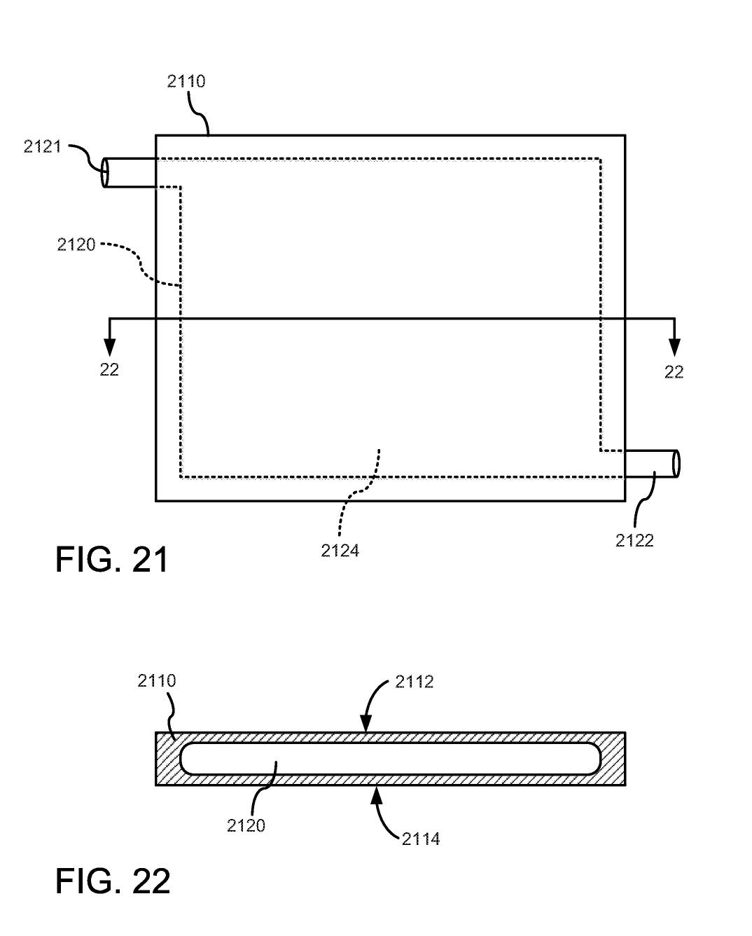

FIGS. 21 and 22 illustrate one exemplary heating mechanism that can be used in embodiments of the disclosed technology. In particular, FIG. 21 is a top view of a pad 2110, and FIG. 22 is a cross-sectional side view of the pad 2110. The pad 2110 of FIGS. 21 and 22 is similar to that of housing 1910 but includes one or more conduits for circulating a fluid (e.g., a liquid or gas, such as air) through one or more interior spaces of the heating pad 2110. In the illustrated embodiment, for example, a first conduit 2120 extends into and includes a central chamber 2124 in the interior of the housing 2110. The first conduit 2120 additionally includes an inflow side 2121 and an outflow side 2122. Although only a single conduit is shown in FIGS. 21 and 22, one or more additional conduits can be manufactured into the heating pad 2110. Furthermore, instead of comprising a central chamber, the first conduit 2120 can wind through the interior of the heating pad 2110 as in the housing 1710 of FIG. 17. The heating pad 2110 can be formed to include suitable spaces or channels for the one or more conduits. The walls of the conduits can be formed from a variety of materials, as described above with respect to FIG. 10-11. Alternatively, in certain embodiments, the one or more conduits do not have a separate conduit wall but are instead formed directly from the material from which the housing 2110 is manufactured.

As best shown in the cross-sectional view of FIG. 22, the body of the first conduit 2120 is located between a bottom surface 2114 of the heating pad 2110 and a top surface 2112. In this embodiment, because the TMS coil is placed on the top surface 2112 of the pad, there is no interior cavity configured to enclose the TMS coil. The first conduit 2120 can be used to circulate a heated or cooled fluid through the central chamber 2124 of the pad 2110, thereby changing the temperature of the bottom surface 2114 of the pad to a desired temperature. To supply the heated or cooled fluid, the first conduit 2120 can be fluidly coupled to a variety of pumping or forced-air systems with heating and/or cooling capabilities. In use, the first conduit 2120 can be used to alter the temperature of a subject's cranium (and thus the cells in the subject's cerebral cortex) prior to or during TMS treatment. One or more thermal sensors (e.g., a thermistor or other temperature sensing device) can be affixed to the bottom surface of the pad 2110 or placed on the scalp of the subject at or near the region being treated in order to monitor the temperature. The circulation and temperature of the fluid in the first conduit 2120 can then be adjusted as appropriate in order to achieve the desired temperature changes.

In further embodiments, the device used to increase or decrease the temperature of the subject's cranium has the form of a hat, helmet, or bonnet that is fluidly coupled to a temperature controlled air supply. In some instances, the heating or cooling is desirably performed in the absence of TMS and while the subject's head is in an MRI machine (e.g., in order to obtain baseline measurements or temperature change distributions), in which case the conditioned air may be ducted via an MRI-compatible plastic hose. The temperature of the conditioned air can be monitored. For instance, the ducts supplying the air can be monitored using one or more embedded thermocouples. In certain embodiments, the temperature of the air can be limited by using a thermal fuse designed to shut down the power to the heating or air conditioning unit should the temperature exceed a desired maximum or minimum temperature. (e.g., if the temperature is below 42° C.). In certain implementations, to provide cold-conditioned air, room air can be blown across a liquid-to-air heat exchanger that is supplied with cold water from a standard laboratory closed loop water bath. Suitable condensate traps can be installed in the supply line to prevent any liquids from travelling to the MRI unit. To provide warm conditioned air, air can be supplied by a commercial hair dryer outfitted with a thermostat, thermocouple, and thermal fuse described above as well as a sufficient length of hose to reach the subject. Or, in other implementations, a heat exchanger can be used.

C. Exemplary Methods of Use

Any of the embodiments disclosed above can be used to provide heating (or cooling) to a subject's cranium before, during, and/or after tDCS or TMS treatment. For ease of illustration, the discussion below refers to exemplary treatment methods involving the heating of a subject's cranium before, during, and/or after tDCS or TMS treatment. It is to be understood, however, that any of the described methods can involve cooling of a subject's cranium instead. Furthermore, while any of the disclosed methods can be performed using the tDCS or TMS devices described herein, they can also be performed using conventional tDCS, TMS, or other magnetic or electrical stimulation systems.

In one exemplary embodiment, the subject's cranium is heated prior to tDCS or TMS treatment, thereby elevating the temperature of cells in a target region of the subject's cerebral cortex to an elevated temperature. In particular embodiments, the elevated temperature is sufficient to create adenosine and cAMP levels that trigger the release of intracellular calcium. The heat can be applied for any period of time before application of the tDCS treatment. In particular implementations, however, the heat is applied for 30 minutes or less, such as between 10 and 20 minutes (e.g., about 15 minutes). In some implementations, however, the heat is applied for only 10 minutes or less, such as 5 minutes or less. After the heat is applied, the heating elements of the housing can be deactivated, or, in some embodiments, operated to return the region of interest of the subject (e.g., the region of the cerebral cortex being stimulated) to its base line or normothermic temperature (e.g., about 37° C.) or to any lower temperature. For example, in embodiments in which the heating elements comprise conduits for circulating water, cool water can be circulated after the desired period of heating. In some embodiments, tDCS or TMS treatment can begin immediately after the heat is removed or after some period of time (e.g., after a period of 30 minutes or less, 10 minutes or less, or 5 minutes or less). In other embodiments, heating is continued throughout at least a portion of the tDCS or TMS treatment. For example, the heating can continue during the tDCS or TMS treatment, and both treatments can be simultaneously or substantially simultaneously discontinued. In still further embodiments, the heating can continue during tDCS or TMS treatment and continue beyond the tDCS or TMS treatment. In further embodiments, the heating is staggered with the tDCS or TMS treatments, such that a period of heating is followed by a period of tDCS or TMS without heating, which is then followed by another period of heating, and so on. This sequence of staggered heat application and tDCS or TMS application can continue through any number of heating or tDCS or TMS cycles.

In other embodiments, heating is not applied before the tDCS or TMS treatment. For example, in some embodiments, the heating is performed during the tDCS or TMS treatment. For instance, the heating can be performed synchronously with the tDCS or TMS treatment or can be activated at some time after the tDCS or TMS treatment has begun. As above, the heating can continue after the tDCS or TMS treatment is complete to further promote long-term potentiation or can be discontinued simultaneously with or before completion of the tDCS or TMS treatment. In other embodiments, the heating is performed only after a tDCS or TMS treatment is complete. For instance, heating can begin immediately after a tDCS or TMS treatment or after some period of time from the tDCS or TMS treatment (e.g., after a period of 30 minutes or less, 10 minutes or less, or 5 minutes or less).

The temperature to which the region of interest in the subject's cerebral cortex is heated can vary. In particular implementations, the region of interest is heated to a temperature that is less than 40° C. to avoid hyperthermia. However, in some implementations, the temperature is raised above 40° C., but only for a brief period of time. In particular embodiments, the region of interest in the subject's cranium (e.g., the region of the cerebral cortex being stimulated) is raised to a temperature between 37.5 and 39° C., which is sufficient to trigger the desired head-induced potentiation. The rate of temperature change can also be monitored and regulated during application of the heat. In particular implementations, the rate of temperature change is between 2 and 5° C./minute (e.g., substantially 3.5° C./minute). It should be understood, however, that other rates are possible, including faster rates of change or slower rates of change.

To accomplish the desired temperature and rates of temperature change in the cells of the subject's cerebral cortex, the heating elements in embodiments of the heating devices described above may need to be heated to a temperature above the target temperature. Similarly, the heat at the bottom surface of the housing may need to exceed the target temperature in order to effectively heat the region of interest, which is typically one of the regions of the subject's cerebral cortex. However, as more fully explained below, the scalp thickness and cerebral blood flow in a typical human allow the scalp to be an effective conductor of heat to the underlying dura and brain tissue. Significant temperature gradients take place near the brain surface and exponentially decrease with distance from the brain surface according to a characteristic shielding length (hereafter denoted “Δ”), which is inversely proportional to the square root of cerebral blood flow (“CBF”). Typically, CBF in adult humans is about 50 ml/100 g/min, resulting in a Δ of about 3-4 mm. This number is much smaller than the human brain diameter of about 14 cm, meaning that the temperature in the adult human brain is largely homogeneous except for a narrow (˜Δ) shell near the brain surface. Thus, assuming a temperature of 39° C. at the surface of the subject's scalp and a temperature of 37° C. at a depth of 4 mm, the temperature gradient is 2° C. per 4 mm, or 0.5° C./mm. The depth of the cerebral cortex, which is the target of tDCS and TMS, is about 1.5 mm. Thus, with a temperature of 39° C. at the surface of the subject's scalp, the region of interest in the cortex should experience temperatures between 39 and 37.5° C., which are sufficient to trigger adenosine release and temperature-induced potentiation. Furthermore, the average diameter of the head of adult humans is ˜15 cm. As blood flow in the scalp is less than that in the cerebral cortex and typically about 10 ml/100 g/min, the corresponding shielding length Δ is about 7 mm. This value is much larger than the scalp thickness, which is typically about 1-2 mm. Accordingly, the scalp effectively conducts a temperature applied to the exterior surface of the scalp (e.g., from the bottom surface of any of the heating devices described above) to the underlying dura and brain tissue.

As noted above, any of the treatment methods described above can alternatively involve cooling of a subject's cranium rather than heating. In such embodiments, the cooling process can be performed for similar durations as the heating embodiments and can be applied in a similar manner as the heating embodiments relative to the tDCS or TMS treatment (e.g., the cooling can be applied before, during, or after tDCS or TMS treatment). Furthermore, in some embodiments, rapid cooling can be performed after a period of desired heating, or vice versa. For example, in some embodiments, the subject's cranium can be cooled before heating is applied. For example, the subject's cranium can be cooled for 30 minutes or less, such as between 10 and 20 minutes (e.g., about 15 minutes). The subject's cranium can be cooled, for example, to between 32 and 36° C. After some period of time, the cooling can be discontinued, and the subject's cranium can be heated, either back to its normal temperature range or to an elevated temperature as explained above. The heating can be applied in any of the manners described herein (e.g., before, during, and/or after tDCS or TMS treatment). Embodiments that use both cooling and heating can effectively create a larger temperature change in the brain, which may further affect the synaptic activities in the subject's cerebral cortex.

In still further embodiments, the cooling is performed without any subsequent heating. In such embodiments, the cooling is applied to brain areas adjacent to the tDCS electrode or TMS coil, in order to reduce potentiation in these adjacent brain areas and increase the focality of the therapy to areas directly targeted by the tDCS electrode or TMS coil. For instance, the cooling can reduce neural kinetics and/or increase the synaptic firing threshold. In particular embodiments, focality is achieved by cooling areas that neighbor the area targeted by tTDCS or TMS and using a tDCS electrode or a TMS coil (such as an H-Coil or figure-of-8 coil) to stimulate deep tissue in the area targeted by tDCS or TMS. In other embodiments, cooling is achieved by cooling the surface of the target area, while tDCS or TMS is used to stimulate the deep target area without affecting the surface tissue. For instance, the cooling of the surface tissue of the target area will reduce the TMS potentiation at the surface and for a depth beneath the tissue (e.g., at least 1 cm below the surface). Consequently, the stimulation at the deeper target area can be more focalized.

In certain embodiments in which a subject's cranium is cooled, a portion of the cranium above the target area is cooled using one or more ice packs (or other cooling mechanism) for a period of time (e.g., between 10-180 minutes, such as between 60-120 minutes). TMS is then applied. TMS can be applied during the cooling process, after the cooling process, or both during and after the cooling process. For instance, single pulse TMS can be applied (e.g., at a sampling interval of once per 2-10 seconds, such as once per 5 seconds) and/or rPAS can be applied. For instance, both single pulse TMS and rPAS stimulation can be provided (e.g., sequentially in any order). TMS and/or rPAS can be applied for any duration (e.g., for any of the durations discussed above). Furthermore, the pulse trains used during TMS and/or rPAS can vary widely in frequency, duration, and signal shape.

In certain specific implementations, methods for producing motor-evoked potentials (“MEPs”) from TMS or rPAS are performed in combination with the cooling of a subject's scalp. Such methods can be used, for example, to study the effects of temperature on TMS. In some implementations, magnetic stimulation is delivered using a figure-of-8 coil (e.g., a 9.5 cm external diameter coil) connected to a stimulator. Stimulation can be applied over the scalp region of a subject's right first dorsal interosseus muscle (“FDI”) in order to find one or more sites on the scalp that will yield the strongest FDI motor-evoked potentials at a given suprathreshold intensity (also referred to as “motor hot spots”). These motor hot spots can be determined by moving the coil over the hand motor area while the subject relaxes his/her arm muscles. Once a hot spot is found, a resting motor threshold (“rMT”) can be found that corresponds to the minimum stimulation intensity over the motor hot spot that can elicit an MEP response (e.g., the intensity at which an MEP of greater than 50 μV is measured for at least some percentage (such as 50%) of contractions of the contralateral FDI). To potentiate activity of the identified motor cortical areas, an rTMS pulse train can be generated (e.g., a 1-to-300-second-long rTMS pulse train, such as a 160-second long pulse train set at 5 Hz). Further, in certain implementations, each rTMS pulse is paired with an electrical conditioning stimulus given to the right median nerve. The inter-stimulus interval between the electrical conditioning stimulus can vary (e.g., between 1-100 ms, such as 25 ms). The intensity of the stimuli can also vary, but in certain implementations is set at between 60-200% (such as 90%) of the participant's rMT. The intensity of the electrical conditioning stimulus can be varied as well, but in certain implementations is set to greater than the sensory threshold (such as twice the sensory threshold). Electromyography can be performed to evaluate the electrical activity at the stimulation region. For example, EMG electrodes can be placed on the right first dorsal interosseous muscle in a bipolar montage. The EMG signals can be amplified using an EMG machine with a suitable bandpass range (e.g., between 10 and 1000 Hz). The resulting signal can then be digitized (e.g., at a frequency of 5 kHz or other suitable frequency) and input into a computer for off-line analysis. To help stimulate the appropriate areas, navigated TMS can be used. For example, an rTMS neuronavigation system can be used. Such systems are typically based on frameless stereotaxy and avoid fixing the head in a stereotactic ring. In certain embodiments, such systems combine MRI with TMS using a 3D digitizer to measure the position of the coil and map this position onto the MRI data set. The stimulated brain area can then be visualized on a computer screen during the stimulation.

With this arrangement and with the subject's rMT identified, the subject's scalp can be cooled for some period of time (e.g., between 10-180 minutes, such as between 60-120 minutes). For instance, the subject's scalp can be cooled to a target temperature (e.g., 34° C.). During the cooling period, the time course of MEP changes can be sampled by evoking a TMS response (e.g., periodically, such as every 5 seconds). After the cooling period, the scalp cooling pack can be removed and the amplitude of the motor evoked potential response to TMS targeting motor cortex can be measured (e.g., periodically, such as every 5 seconds). Further, rPAS can also or alternatively be performed. Baseline measurements can also be generated. For instance, TMS and/or rPAS can be performed in the absence of cooling in order to obtain baseline MEP measurements. Also, cooling can be performed in the absence of TMS or rPAS while the subject is in an MRI coil in order to measure temperature changes and distribution in the brain.

As noted above, embodiments of the disclosed technology can be used during magnetic resonance imaging (“MRI”) scanning in order to help reduce the risk of heating, aberrant current flow, and/or device movement in patients with medical device implants. For example, a patient may have a deep brain stimulator or metal plate implanted in their cranium. The pulsed RF fields that are used during an MRI scan can create undesirable currents, heat, or vibrations in the device or plate, potentially causing cell death and brain damage. To help reduce or otherwise mitigate these effects, the temperature of the patient's cranium can be modulated (e.g., cooled) using any of the suitable embodiments disclosed herein. For example, any of the temperature modulation devices disclosed herein that are MRI compatible can be used to heat or cool a patient's cranium before and/or during an MRI scan. Further, non-MRI-compatible devices can be used to cool or heat a patient's cranium before the MRI scan.

In general, cooling before and/or during an MRI scan can be used to reduce the temperature rise induced in the brain due to pulsed RF Fields. The reduced neural response to electrical stimulation that results from cooling can also be used to prevent exitotoxic cell death caused by aberrant current flow induced through an implantable electrical stimulator in response to an MRI scan. MRI scanning can also cause implants to vibrate in response to pulsed magnetic fields. Cooling can serve as a neuroprotectant preventing cell death that can occur due to implant vibration.

As also noted above, embodiments of the disclosed technology can be used for diagnostic purposes. In certain implementations, for example, a TMS coil or other magnetic, electric, or other stimulator can be operated (e.g., using a single non-repeating pulse, a pulse pair, or repeating pulses) to evoke potentiations in a patient's brain. The potentiations can be motor-evoked potentials or other potentials detected using electromyography, electroencephalography, and/or another suitable detection technique. The resulting measurements can then be used for diagnostic purposes. For instance, evoked potentiations generated from the brain can be used as an indicator of aberrant brain function (e.g., caused by a wide variety of potential diseases of the brain). For example, the responses can be compared to baseline or expected responses in order to detect aberrations. The location and degree of the aberration can then be used to help diagnose a potential disease or injury to the patient's brain.

In certain embodiments, a normative database of evoked potentiations can be created and used as part of the diagnostic procedure. The database can comprise, for example, responses from a plurality of subjects that are considered to be normal as well as responses from subjects that are indicative of certain diseases, injuries, or other neural conditions. By correlating observed responses to the data from the normative database, a disease, injury, or neural condition can be diagnosed. In order to enhance the diagnosis by creating a larger dataset of evoked responses, the stimulation and measurement of the potentiation response can be performed while a patient's cranium is modulated to a variety of different temperatures. The various temperatures can be achieved and maintained, for instance, using embodiments of the disclosed technology.

In some embodiments, a patient's evoked potentiation are measured and analyzed at different periods of time. For instance, evoked potentiations in a patient can be detected at two or more different times in order to measure disease progression or treatment efficacy. Again, the stimulation and measurement of the evoked potentials can be performed while a patient's cranium is at a variety of different temperatures using embodiments of the disclosed technology.

Further, certain neurotransmitters are understood to react differently to magnetic or electrical stimulation than other neurotransmitters when the brain is cooled or heated. Consequently, by cooling or heating a patient's cranium and subsequently analyzing evoked potentiations, the reaction of certain neurotransmitters to stimuli can be better isolated and targeted. Deficiencies or unusual abundances of the targeted neurotransmitter can then detected and used for diagnostic purposes (e.g., to diagnose depression or other neural conditions).

Another use of temperature modulation is to monitor the changes in neurotransmitters as a result of treatment (e.g., as a result of a drug treatment). For example, a patient can be monitored at various times during the course of treatment to detect whether a particular treatment is having its intended effect on the targeted neurotransmitters.

Having illustrated and described the principles of the disclosed technology, it will be apparent to those skilled in the art that the disclosed embodiments can be modified in arrangement and detail without departing from such principles. For example, although the systems, apparatus, and methods are described as being primarily applied to a subject's scalp for potentiation or synaptic modulation of a region of the subject's cerebral cortex, any of the embodiments disclosed herein can be used to treat other regions of a subject. The flexible pad-shaped embodiments, for example, can be placed against numerous surfaces of a subject to be treated, including surfaces that are not easily accessible. For example, any of the systems can be used to treat hard-to-reach regions of patients who are at least partially immobile. Furthermore, any of the housings with heating elements described herein can be used alone, in the absence of a tDCS or TMS device in order to create heat-induced potentiation without complementary tDCS or TMS stimulation.

In view of the many possible embodiments to which the principles of the disclosed technologies can be applied, it should be recognized that the illustrated embodiments are only preferred examples of the technologies and should not be taken as limiting the scope of the invention. Rather, the scope of the invention is defined by the following claims and their equivalents. We therefore claim all that comes within the scope and spirit of these claims.

1-48. (canceled)

49. A method, comprising:
for a first period of time,
decreasing a temperature of a region of the cranium of a subject from a base line temperature to a cooled temperature, the region of the cranium of the subject being adjacent to a target region of the cerebral cortex of the subject, and
sustaining the cooled temperature at the region on the cranium of the subject; and for a second period of time,
activating an electrical or magnetic stimulator positioned at or adjacent to the region of the cranium of the subject, thereby electrically or electromagnetically stimulating the target region of the cerebral cortex of the subject; and
sustaining the activation of the electrical or magnetic stimulator.

50. The method of claim 49, wherein the first period of time ends before the second period of time begins.

51. The method of claim 49, wherein the first period of time and the second period of time at least partially overlap.

52. The method of claim 49, wherein the second period of time ends before the first period of time begins.

53. The method of claim 49, wherein the method further comprises, increasing the temperature of the region of the cranium of the subject from the cooled temperature before the activating of the electrical or magnetic stimulator.

54. The method of claim 49, wherein the method further comprises increasing the temperature of the region of the cranium of the subject from the cooled temperature while the electrical or magnetic stimulator are activated.

55. The method of claim 49, wherein the method further comprises increasing the temperature of the region of the cranium of the subject from the base line temperature to an elevated temperature before the first period of time.

56. The method of claim 49, wherein the decreasing the temperature of the region of the cranium of the subject and the activating of the electrical or magnetic stimulator are performed concurrently.

57. The method of claim 49, wherein the decreasing the temperature of the region of the cranium of the subject comprises circulating a fluid at or below the cooled temperature through conduits in a flexible housing positioned on the region of the cranium of the subject.

58. The method of claim 49, performed as part of a treatment for depression, stroke recovery, Parkinson's disease, Alzheimer's disease, autism, post-traumatic stress disorder (“PTSD”), addictive behavior, anxiety, dysthymia, dystonia, epilepsy, pain, obsessive compulsive disorder, or schizophrenia.

59-72. (canceled)

73. A method, comprising:
decreasing a temperature of a region of the cranium of a subject from a base line temperature to a cooled temperature, the region of the cranium of the subject being adjacent to a device implanted in the subject; and
performing a magnetic resonance imaging scan of the cranium of the subject.

74. The method of claim 73, further comprising maintaining the cooled temperature of the cranium of the subject during at least a portion of the magnetic resonance imaging scan.

75. The method of claim 73, wherein the cooled temperature is maintained for a period sufficient to cause cells in the cerebral cortex of the subject to have a reduced reaction to stimuli produced by the magnetic resonance imaging.

76. The method of claim 73, wherein the device implanted in the subject is a implanted brain stimulator.

77. A method, comprising:
causing the temperature of a subject's cranium to be changed from a normothermic temperature to a modulated temperature above or below the normothermic temperature, wherein the temperature change is induced by a heating or cooling device placed on the subject's cranium;
detecting a set of one or more electrical potentials in the subject's brain or muscles while the subject's cranium is at the modulated temperature, the electric potentials being evoked by a set of one or more electrical or magnetic stimuli; and
performing a diagnostic procedure using the detected set of one or more electrical potentials.

78. The method of claim 77, wherein the set of one or more electrical potentials comprises a first set of one or more electrical potentials, wherein the modulated temperature is a first modulated temperature, wherein the set of one or more electrical or magnetic stimuli comprises a first set of one or more electrical or magnetic stimuli, and wherein the method further comprises:
causing the temperature of a subject's cranium to be changed from the first modulated temperature to a second modulated temperature;
detecting a second set of one or more electrical potentials in the subject's brain or muscles while the subject's cranium is at the second modulated temperature, the second set of electric potentials being evoked by a second set of one or more electrical or magnetic stimuli; and
performing the diagnostic procedure using the detected first and second sets of electrical potentials.

79. The method of claim 77, wherein the modulated temperature is above the normothermic temperature.

80. The method of claim 77, wherein the modulated temperature is below the normothermic temperature.

81. The method of claim 77, wherein the heating or cooling device placed on the subject's cranium further comprises a transcranial magnetic stimulator coil or transcranial direct current stimulator.

82. A device for use with a transcranial magnetic stimulator coil or transcranial direct current stimulator system, comprising:
a flexible housing having a top surface and a bottom surface,
the flexible housing defining an interior cavity, the interior cavity being configured to at least partially enclose the transcranial magnetic stimulator coil or an electrode of the transcranial direct current stimulator system,
the flexible housing further including one or more apertures configured to provide access to the interior cavity when the transcranial magnetic stimulator coil or electrode of the transcranial direct current stimulator system is positioned within the interior cavity,
wherein the flexible housing further comprises one or more heating or cooling elements disposed in a body of the flexible housing, and wherein the one or more heating or cooling elements comprise one or more fluid conduits disposed in the body of the flexible housing.
